(19) United States
(12) Patent Application Publication
(10) Pub. No.: US 2026/0137702 A1
(43) Pub. Date: May 21, 2026

(54) Salicylate Compound Composition

(71) Applicant: Innovate (EU) Limited, Manchester (GB)

(72) Inventors: James Stuart, Manchester (GB); Simon Cohen, Manchester (GB); Jan Cohen, Manchester (GB)

(21) Appl. No.: 19/389,155

(22) Filed: Nov. 14, 2025

Related U.S. Application Data
(60) Provisional application No. 63/720,982, filed on Nov. 15, 2024.

(51) Int. Cl. A61K 31/616; A61K 47/14; A61K 47/22; A61P 43/00

(52) U.S. Cl. CPC A61K 31/616; A61K 47/14; A61K 47/22; A61P 43/00

(57) ABSTRACT

A liquid composition contains a salicylate compound (e.g. aspirin), glycerin triacetate, saccharin. The salicylate compound is soluble in the composition, which is particularly suitable for oral, parenteral or pulmonary administration.

This application claims priority from U.S. 63/720,982, filed 15 Nov. 2024, the contents and elements of which are herein incorporated by reference for all purposes.

### FIELD OF INVENTION

The present invention relates to a stable salicylate compound composition, e.g. aspirin composition, in liquid form, in particular which is suitable for oral use, intravenous administration or inhalation.

### BACKGROUND

Animal productivity refers to the ability for livestock and other commercially raised animals to survive and thrive under conditions where maximum economic benefit is achieved. Productivity can be assessed by measurement of production traits including animal weight, milk production, egg laying capacity, reproductive turnover etc.

For animals raised for the commercial production of meat, such as beef cattle, pigs or poultry, a goal may be to maximize the production of quality meat while minimizing the cost, such as, feed costs, associated with raising the animals. A key factor in optimising animal productivity is diet, with numerous feed additives being identified that support various production traits, as discussed in Al-Jaf et al. International Journal of Agriculture and Forestry. (2019) 9(1):16-31. However, with increasing economic pressure on the livestock industry, there remains a need to identify further means of optimising production and performance in commercially raised animals for sustained economic viability.

Aspirin (acetylsalicylic acid) has been widely used as an analgesic to relieve minor aches and pains, as an antipyretic to reduce fever, and as an anti-inflammatory medication. Aspirin also has a ‘COX’ mediated antiplatelet effect by inhibiting the production of thromboxane, which under normal circumstances binds platelet molecules together to create a patch over damaged walls of blood vessels. Because the platelet patch can become too large and also block blood flow, locally and downstream, aspirin is also used long-term, at low doses, to help prevent heart attacks, strokes, and blood clot formation in people at high risk of developing blood clots. Also, low doses of aspirin may be given immediately after a heart attack to reduce the risk of another heart attack or the death of cardiac tissue.

The main drawback of aspirin is its ability to cause gastric mucosal damage, which is exacerbated by the presence of particulate aspirin. Lan et al. (supra) identified these side-effects as the limiting factor in the treatment of glioma with aspirin. Fully solubilised, particulate free aspirin would be expected to reduce the local cytotoxic irritant effect of aspirin.

All traditional ‘stable’ aspirin formulations, whether they are called dispersible, soluble or effervescent, are simply dispersible particle formats, which will settle out into particulate form soon after being dissolved.

A number of studies have demonstrated that the direct topical irritation of aspirin particles on the gastric mucosa causes the damage leading to the side effects seen when taking oral aspirin [Kevin et al., ‘Acute effect of systemic aspirin on gastric mucosa in man’, Digestive Diseases and Sciences, 1980, Vol. 25, Issue 2, pp 97-99; Cooke et al., ‘Failure of intravenous aspirin to increase gastrointestinal blood loss’, British medical journal, 1969; Vol. 3(5666), pp. 330-332; Wallace et al., ‘Adaptation of rat gastric mucosa to aspirin requires mucosal contact’ Am J Physiol, 1995, Vol. 268, G134-8; Cryer et al. ‘Effects of low dose daily aspirin therapy on gastric, duodenal and rectal prostaglandin levels and on mucosal injury in healthy humans’, Gastroenterology, 1999, Vol. 117, pp. 17-25; Lichtenberger et al., ‘Where is the evidence that cyclooxygenase inhibition is the primary cause of nonsteroidal anti-inflammatory drug (NSAID)-induced gastrointestinal injury?Topical injury revisited’, Biochemical Pharmacology, 2001, Vol. 61, pp. 631-637; Ligumsky M et al., ‘Aspirin can inhibit gastric mucosal cyclo-oxygenase without causing lesions in the rat’, Gastroenterology, 1983, Vol. 84, pp 756-61; and Zhao et al., ‘Clinical Research Feasibility of intravenous administration of aspirin in acute coronary syndrome’, Journal of Geriatric Cardiology, 2008, Vol. 5 No. 4, pp. 212-216, all incorporated herein by reference]. It follows that if the particles are removed by producing a stable, fully solubilised liquid aspirin, the adverse GI side effects will be significantly reduced or even totally eliminated.

The development of such an improved liquid composition is demonstrated in WO2016102959A1, which is hereby incorporated by reference. Such an improved liquid composition is associated with reduced gastric mucosal damage, and high tolerability.

The present invention has been devised in light of the above considerations.

### SUMMARY OF THE INVENTION

In one aspect of the invention there is provided a method of enhancing a production trait of an animal, the method comprising administering to the animal an effective amount of a liquid composition comprising a salicylate compound, glycerin triacetate, and saccharin, wherein the salicylate compound is selected from the group consisting of aspirin, triflusal, and diflunisal, salsalate and salicylic acid, wherein the composition comprises 0.1 to 7 wt % salicylate compound and 90 to 99.9 wt % glycerin triacetate.

In one aspect of the invention there is provided a liquid composition for use in a method of enhancing a production trait of an animal, the liquid composition comprising a salicylate compound, glycerin triacetate, and saccharin, wherein the salicylate compound is selected from the group consisting of aspirin, triflusal, and diflunisal, salsalate and salicylic acid, wherein the composition comprises 0.1 to 7 wt % salicylate compound and 90 to 99.9 wt % glycerin triacetate.

In one aspect of the invention there is provided the use of a liquid composition in a method of enhancing a production trait of an animal, wherein the liquid composition comprises a salicylate compound, glycerin triacetate, and saccharin, wherein the salicylate compound is selected from the group consisting of aspirin, triflusal, and diflunisal, salsalate and salicylic acid, wherein the composition comprises 0.1 to 7 wt % salicylate compound and 90 to 99.9 wt % glycerin triacetate.

In one aspect of the invention there is provided the use of a liquid composition in the manufacture of a medicament for use in a method of enhancing a production trait of an animal, wherein the liquid composition comprises a salicylate compound, glycerin triacetate, and saccharin, wherein the salicylate compound is selected from the group consisting of aspirin, triflusal, and diflunisal, salsalate and salicylic acid, wherein the composition comprises 0.1 to 7 wt % salicylate compound and 90 to 99.9 wt % glycerin triacetate.

The liquid composition provides a stable, liquid form of fully solubilised salicylate compound. As a result, the GI irritant side-effects discussed above are dramatically reduced, avoided, ameliorated or eliminated. Furthermore, the liquid form provides a very easily administered composition.

In some embodiments, the production trait is selected from growth rate, weight gain, milk production, and/or egg production. In preferred embodiments, the production trait is weight gain of an animal.

In some embodiments, the animal is a non-human animal. The animal may be is a livestock animal. The animal may be selected from the group comprising: poultry, cattle, pigs and sheep. The animal may be a monogastric animal. The animal may be a mammal or a bird. The mammal may be a chicken. The animal may be a pig.

In some embodiments, the method is a non-therapeutic method.

The salicylate compound may be selected from the group consisting of: aspirin, triflusal, diflunisal, salsalate and salicylic acid. In preferred embodiments the salicylate compound is aspirin.

In some embodiments the composition may comprise or consist of aspirin, glycerin triacetate, saccharin and a flavouring agent.

In some embodiments, the composition comprises 0.1 to 5 wt % salicylate compound, optionally wherein the composition comprises 0.125 to 3 wt % salicylate compound. In some embodiments, the composition comprises 0.125% wt % salicylate compound. In some embodiments, the composition comprises 0.25 wt % salicylate compound. In some embodiments, the composition comprises 2.5 wt % salicylate compound.

In some embodiments, the composition comprises 94 to 99.9 wt % glycerin triacetate. In some embodiments, the composition comprises 95 to 99.875 wt % glycerin triacetate.

In some embodiments the concentration of the salicylate compound, preferably aspirin, may be 0.1 to 5 wt % and/or the concentration of glycerin triacetate may be 94 to 99.9 wt %. In some embodiments, the composition comprises 0.125 to 3 wt % salicylate compound and 94 to 99.875 wt % glycerin triacetate. In some embodiments the concentration of saccharin may be 0.1 to 3 wt %.

For the avoidance of doubt, the components are present in the composition such that the total amount is equal to 100 wt %. For example, for a composition consisting of salicylate compound, glycerin triacetate and saccharin, there may be 0.1 to 3 wt % salicylate compound, preferably aspirin, and 0.1 to 3 wt % saccharin. The glycerin triacetate will then make up the balance of the composition to 100 wt %.

In some embodiments, the composition comprises 0 to 0.5 wt % water.

In some embodiments, the liquid composition further comprises a flavouring agent, optionally wherein the flavouring agent is mint oil.

The glycerin triacetate may be obtained or obtainable by a purification process. This process may be suitable to remove water or other impurities from the glycerin triacetate. For example, the glycerin triacetate may be treated by distillation and/or by passing through activated earth. The glycerin triacetate may be obtained or be obtainable by passing through activated earth.

In some embodiments the liquid composition may have a salicylate compound, e.g. aspirin, degradation rate at 25° C. of less than 0.04%/day and/or less than 0.006 mg/g/day. In some embodiments the composition may have a salicylate compound, e.g. aspirin, degradation rate at 25° C. of less than 0.02%/day and/or less than 0.004 mg/g/day.

The liquid composition may be free of particulates. The salicylate compound may be completely soluble in the liquid composition. In some embodiments, greater than 90% of the total amount of salicylate compound, preferably aspirin, in the composition is fully dissolved, for example at least 95%, at least 96%, at least 97%, at least 98%, at least 99% or at least 99.9%. In some embodiments 100% of the total salicylate compound is fully dissolved.

In some embodiments, the liquid composition is suitable for oral use, formulated for intravenous or intra-arterial administration, or formulated for inhalation or insufflation administration.

In another aspect of the present invention a packaged article comprising the liquid composition of the present invention is sealed therein is provided. The packaged article may be a bottle, pipette, syringe, vial, sachet, stick shot and liquid gel capsule.

The Blow-Fill-Seal (BFS) method may be used to manufacture the packaged article.

In some embodiments the method of enhancing a production trait of an animal involves oral, rectal, nasogastric, parenteral (e.g. intravenous or intrarterial), inhalation or insufflation administration of the liquid composition.

### DESCRIPTION

The inventors have discovered that by formulating a salicylate compound together with a glycerol derivative and a saccharin compound that a highly stable liquid salicylate formulation can be provided.

### Salicylate Compound

The term “salicylate compound” as used herein refers to compounds according to Formula (I):

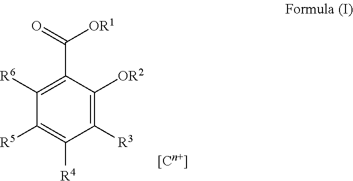

wherein:
R¹ is a negative charge, or is independently selected from the group consisting of
   —H,
   —R^{A}
   —OH, —OR^{A}, —CF₃, —OCF₃,
   —COH, —COR^{A}, —COOH, —COOR^{A},
   —NH₂, —NHR^{A}, —NR^{A}₂ and —NR^{B}₂;
R² is independently selected from the group consisting of
   —H,
   —R^{A},
   —OH, —OR^{A}, —CF₃, —OCF₃,
   —COH, —COR^{A}, —COOH, —COOR^{A},
   —NH₂, —NHR^{A}, —NR^{A}₂, —NR^{B}₂, and
   -Q;
R³-R⁶ are each independently selected from the group consisting of
   —H,
   —F, —Cl, —Br, —I,
   —R^{A},
   —OH, —OR^{A}, —CF₃, —OCF₃,
   —CN, —NO₂,
   —COH, —COR^{A}, —COOH, —COOR^{A},
   —NH₂, —NHR^{A}, —NR^{A}₂, —NR^{B}₂,
   —SO₃H, —S(O)R^{A} and —S(O₂)R^{A};
Q is

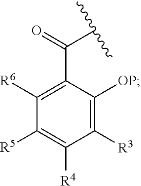

and P is independently selected from —H, linear or branched C₁₋₄alkyl, alkenyl or alkynyl, or —COR^{A};
wherein —R^{A} is independently selected from the group consisting of
   linear or branched C₁₋₄alkyl, alkenyl or alkynyl,
   phenyl optionally substituted with one or more groups —R^{D},
   benzyl optionally substituted with one or more groups —R^{D},
   —COOH, —COOR^{C}, —C(O)R^{C}, —NH₂, —NHR^{C} or —NR^{C}₂;
—R^{D} is independently selected from
   linear or branched C₁₋₄alkyl, alkenyl or alkynyl,
   —F, —Cl, —Br, —I,
   —OH, —OR^{C}, —CF₃, —OCF₃,
   —CN, —NO₂,
   —COH, —COR^{C}, —COOH, —COOR_{C},
   —NH₂, —NHR^{C}, —NR^{C}₂, —NR^{B}₂,
   —S(O)R^{C} and —S(O₂)R^{C};
—R^{C} is independently selected from linear or branched C₁₋₄alkyl, alkenyl or alkynyl;
—NR^{B}₂ is independently selected from the group consisting of azetidino, imidazolidino, pyrazolidino, pyrrolidino, piperidino, piperazino, N—C₁₋₄alkyl-piperazino, morpholino, azepino or diazepino, optionally substituted with one or more groups selected from linear or branched C₁₋₄alkyl, alkenyl or alkynyl, phenyl and benzyl; and
[C⁺] is an optional counter-cation selected from the group consisting of alkali metal ions, alkaline earth metal ions, transition metal ions, Al³⁺, ammonium or substituted ammonium ion and NO₂⁺.

### The Group —R¹

In some embodiments, R¹ is a negative charge, or is independently selected from the group consisting of
—H,
—R^{A}
—COH, —COR^{A}, —COOH, —COOR^{A},
—NH₂, —NHR^{A}, —NR^{A}₂ and —NR^{B}₂.

In some embodiments, R¹ is a negative charge, or is independently selected from the group consisting of —H and —R^{A}.

In some embodiments, R¹ is a negative charge, or is independently selected from the group consisting of
—H,
linear or branched C₁₋₄alkyl, alkenyl or alkynyl,
phenyl optionally substituted with one or more groups —R^{D},
benzyl optionally substituted with one or more groups —R^{D},
and —C(O)R^{C}.

In some embodiments, R¹ is a negative charge, —H, or linear or branched C₁₋₄alkyl, alkenyl or alkynyl.

In some embodiments, R¹ is a negative charge.

In some embodiments, R¹ is —H.

When R¹ is a negative charge, counter-cation [Cⁿ⁺] is present.

### The Group —R²

In some embodiments, R² is independently selected from the group consisting of
—H,
—R^{A},
—COH, —COR^{A}, —COOH, —COOR^{A}, and
-Q.

In some embodiments, R² is independently selected from the group consisting of —H, —COH, —COR^{A}, and -Q.

In some embodiments, R² is independently selected from the group consisting of —H, —COR^{C}, and -Q.

In some embodiments, R² is independently selected from the group consisting of —H, —C(O)CH3, and -Q.

In some embodiments, R² is independently —H.

In some embodiments, R² is independently —C(O)CH₃.

In some embodiments, R² is independently -Q.

The Groups R³-R⁶

In some embodiments, R³-R⁶ are each independently selected from
—H,
—F, —Cl, —Br,
—CN, —NO₂,
—R^{A},
—OH, —OR^{A}, —CF₃ and —OCF₃.

In some embodiments, R³—R⁶ are each independently selected from
—H,
—F, —Cl,
—R^{A},
—OH, —OR^{A} and —CF₃.

In some embodiments, R³—R⁶ are each independently selected from
—H,
phenyl optionally substituted with one or more groups —R^{D},
and —CF₃.

In some embodiments, R⁴ is independently —CF₃, wherein R³, R⁵ and R⁶ are preferably each independently —H.

In some embodiments, R⁵ is phenyl optionally substituted with one or more groups —R^{D}, wherein —R^{D} is preferably —F, and R³, R⁴ and R⁶ are preferably each independently —H.

In some embodiments, each of R³—R⁶ is independently —H.

In some embodiments, the salicylate compound is aspirin (2-(acetoxy)benzoic acid), according to Formula (Ia):

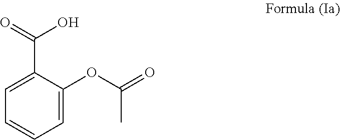

In some embodiments, the salicylate compound is triflusal (2-acetyloxy-4-(trifluoromethyl)benzoic acid), according to Formula (Ib):

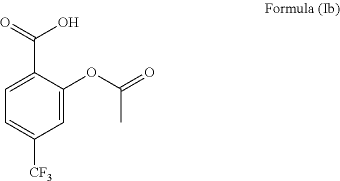 [SMILES: CC(=O)Oc1cc(C(F)(F)F)ccc1C(=O)O]

In some embodiments, the salicylate compound is diflunisal (2′,4′-difluoro-4-hydroxybiphenyl-3-carboxylic acid), according to Formula (Ic):

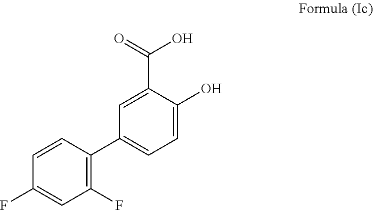 [SMILES: O=C(O)c1cc(-c2ccc(F)cc2F)ccc1O]

In some embodiments, the salicylate compound is salsalate (2-(2-hydroxybenzoyl)oxybenzoic acid), according to Formula (Id):

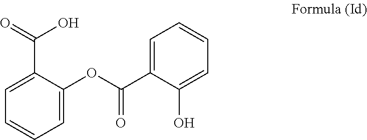 [SMILES: O=C(Oc1ccccc1C(=O)O)c1ccccc1O]

In some embodiments, the salicylate compound is salicylic acid (2-Hydroxybenzoic acid), according to Formula (Ie):

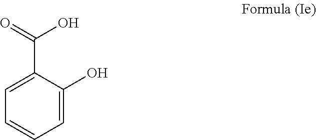

In preferred embodiments the salicylate compound is aspirin.

### Glycerol Derivative

The term “glycerol derivative” as used herein refers to compounds according to Formula (II):

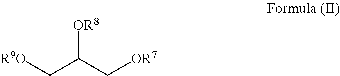

wherein:
R⁷—R⁹ are each independently selected from the group consisting of
   —H,
   —R^{A},
   —COH, —COR^{A}, —COOH, —COOR^{A},
   —NH₂, —NHR^{A}, —NR^{A}₂, —NR^{B}₂,
   —S(O)R^{A} and —S(O₂)R^{A};
wherein —R^{A} is independently selected from the group consisting of
   linear or branched C₁₋₄alkyl, alkenyl or alkynyl,
   phenyl optionally substituted with one or more groups —R^{D},
   benzyl optionally substituted with one or more groups —R^{D},
   —COOH, —COOR^{C}, —C(O)R^{C}, —NH₂, —NHR^{C} or —NR^{C}₂;
—R^{D} is independently selected from
   linear or branched C₁₋₄alkyl, alkenyl or alkynyl,
   —F, —Cl, —Br, —I,
   —OH, —OR^{C}, —CF₃, —OCF₃,
   —CN, —NO₂,
   —COH, —COR^{C}, —COOH, —COOR^{C},
   —NH₂, —NHR^{C}, —NR^{C}₂, —NR^{B}₂,
   —S(O)R^{C} and —S(O₂)R^{C};
—R^{C} is independently selected from linear or branched C₁₋₄alkyl, alkenyl or alkynyl; and
—NR^{B}₂ is independently selected from the group consisting of azetidino, imidazolidino, pyrazolidino, pyrrolidino, piperidino, piperazino, N—C₁₋₄alkyl-piperazino, morpholino, azepino or diazepino, optionally substituted with one or more groups selected from linear or branched C₁₋₄alkyl, alkenyl or alkynyl, phenyl and benzyl.
   The groups R⁷—R⁹

In some embodiments, R⁷—R⁹ are each independently selected from the group consisting of —H and —R^{A}.

In some embodiments, R⁷—R⁹ are each independently selected from the group consisting of —H, linear or branched C₁₋₄alkyl, alkenyl or alkynyl, and —C(O)R^{C}.

In some embodiments, R⁷—R⁹ are each independently selected from the group consisting of —H, and —C(O)R^{C}, wherein R^{C} is preferably methyl.

In some embodiments, one of R⁷—R⁹ is —H and the other two are —C(O)R^{C}, wherein R^{C} is preferably methyl.

In some embodiments, one of R⁷—R⁹ is —C(O)R^{C} and the other two are —H, wherein R^{C} is preferably methyl.

In some embodiments, R⁷—R⁹ are each selected from —C(O)R^{C}, wherein R^{C} is preferably methyl.

In some preferred embodiments, the glycerol derivative is glycerin triacetate (also known as glyceryl triacetate, GTA, or triacetin, or 1,3-Diacetyloxypropan-2-yl acetate) according to Formula (IIa):

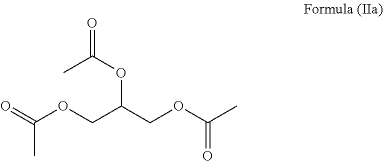

Glycerin triacetate is an FDA approved food additive with “generally regarded as safe” status that has been tested for parenteral nutrition in a wide variety of species with no adverse effects. Glycerin triacetate is also used as a vaporising agent in e-cigarettes and is regarded as safe when used in that context.

The glycerin triacetate (also commonly known as glycerine triacetate, glyceryl triacetate, or triacetin) used herein acts as a solvent for the aspirin or salicylate compound and can give clear aspirin or salicylate compound solutions. Food grade glycerin triacetate may be employed, but it is preferably subjected to further purification processes, e.g. additional distillation steps. In one preferred embodiment, the glycerin triacetate is passed through activated earth, such as a column or fixed bed thereof. The viscosity of glycerin triacetate can be reduced by mixing with a suitable solvent, such as ethanol, prior to passing through the activated earth. The solvent can then be removed by using vacuum distillation followed by steam distillation, preferably to levels below 1 ppm of solvent in the glycerine triacetate.

### Saccharin Compound

The term “saccharin compound” as used herein refers to compounds according to Formula (III):

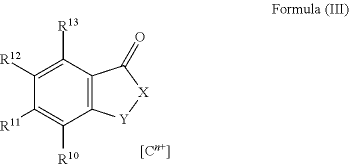

wherein:
R¹⁰—R¹³ are each independently selected from the group consisting of
   —H,
   —F, —Cl, —Br, —I,
   —R^{A},
   —OH, —OR^{A}, —CF₃, —OCF₃,
   —CN, —NO₂,
   —COH, —COR^{A}, —COOH, —COOR^{A},
   —NH₂, —NHR^{A}, —NR^{A}₂, —NR^{B}₂,
   —SO₃H, —S(O)R^{A}, —S(O₂)R^{A}, and
   —W,
X is independently selected from the group consisting of
   —N^{Θ}, —NH and —NR^{A},
Y is independently selected from S(O₂);
wherein —R^{A} is independently selected from the group consisting of
   linear or branched C₁₋₄alkyl, alkenyl or alkynyl,
   phenyl optionally substituted with one or more groups —R^{D},
   benzyl optionally substituted with one or more groups —R^{D},
   —COOH, —COOR^{C}, —C(O)R^{C}, —NH₂, —NHR^{C} or —NR^{C}₂;
—R^{D} is independently selected from
   linear or branched C₁₋₄alkyl, alkenyl or alkynyl,
   —F, —Cl, —Br, —I,
   —OH, —OR^{C}, —CF₃, —OCF₃,
   —CN, —NO₂,
   —COH, —COR^{C}, —COOH, —COOR^{C},
   —NH₂, —NHR^{C}, —NR^{C}₂, —NR^{B}₂,
   —S(O)R^{C} and —S(O₂)R^{C};
—R^{C} is independently selected from linear or branched C₁₋₄alkyl, alkenyl or alkynyl;
—NR^{B}₂ is independently selected from the group consisting of azetidino, imidazolidino, pyrazolidino, pyrrolidino, piperidino, piperazino, N—Cl-4alkyl-piperazino, morpholino, azepino or diazepino, optionally substituted with one or more groups selected from linear or branched C₁₋₄alkyl, alkenyl or alkynyl, phenyl and benzyl;
—W is the group

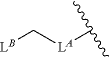

wherein L^{A} is independently selected from a 5- or 6-membered heteroaromatic group and L^{B} is independently selected from a 5- or 6-membered monosaccharide moiety; and
[C⁺] is an optional counter-cation selected from the group consisting of alkali metal ions, alkaline earth metal ions, transition metal ions, Al³⁺, ammonium or substituted ammonium ion and NO₂⁺.
   The group X

In some embodiments, X is independently selected from N^{Θ} and NH.

In some embodiments, X is independently N^{Θ} and [Cⁿ⁺] is a counter-cation selected from alkali metal ions and alkaline earth metal ions.

In some embodiments, X is independently N^{Θ} and [Cⁿ⁺] is a counter-cation selected from sodium and calcium ions.

In some embodiments, X is independently NH.

The groups R¹⁰—R¹³

In some embodiments, R¹⁰—R¹³ are each independently selected from
—H,
—F, —Cl, —Br,
—R^{A},
—OH, —OR^{A}, —CF₃, —OCF₃,
—CN, —NO₂,
—COH, —COR^{A}, —COOH, —COOR^{A},
—NH₂, —NHR^{A}, —NR^{A}₂, —NR^{B}₂, and
—W.

In some embodiments, R¹⁰—R¹³ are each independently selected from
—H,
—F, —Cl, —Br,
—R^{A},
—OH, —OR^{A}, —CF₃, —OCF₃, and
—W.

In some embodiments, R¹⁰—R¹³ are each independently selected from
—H,
—F, —CI,
—R^{A},
—OH, —OR^{A}, and
—W.

In some embodiments, R¹⁰—R¹³ are each independently selected from
—H,
—F, —Cl,
linear or branched C₁₋₄alkyl, alkenyl, alkynyl,
—OH, and
—W.

In some embodiments, R¹⁰—R¹³ are each independently selected from
—H,
—F, —Cl,
linear or branched C₁₋₄alkyl, alkenyl, alkynyl and
—OH.

In some embodiments, one of R¹⁰—R¹³ is independently —W and the remaining groups of R¹⁰—R¹³ are independently —H.

In some embodiments, R¹¹ is independently —W and R¹⁰, R¹² and R¹³ are each independently —H.

The group L^{A}

In some embodiments, L^{A} is a 5- or 6-membered heteroaromatic group independently selected from imidazolyl, pyrazolyl, oxazolyl, isooxazolyl, thiazolyl, isothiazolyl, dioxolanyl, dithiolanyl, triazolyl, furanyl, oxadiazolyl, thiadiazolyl, dithiazolyl, tetrazolyl, pyridinyl, pyranyl, thiopyranyl, diazinyl, oxazinyl, thiazinyl, dioxinyl, dithiinyl, triazinyl and tetrazinyl.

In some embodiments, L^{A} is a 5-membered heteroaromatic group independently selected from imidazolyl, pyrazolyl, oxazolyl, isooxazolyl, thiazolyl, isothiazolyl, dioxolanyl, dithiolanyl, triazolyl, furanyl, oxadiazolyl, thiadiazolyl, dithiazolyl and tetrazolyl.

In some embodiments, L^{A} is a 5-membered heteroaromatic group independently selected from triazolyl, furanyl, oxadiazolyl, thiadiazolyl and dithiazolyl.

In some embodiments, L^{A} is a 5-membered heteroaromatic group independently selected from one of the following triazolyl moieties:

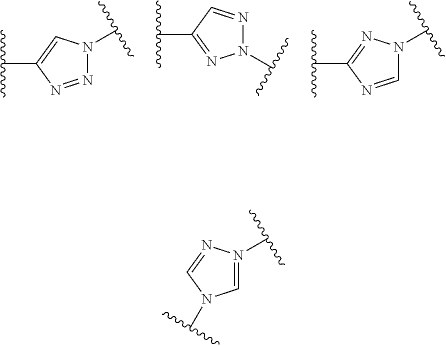

In some embodiments, L^{A} is

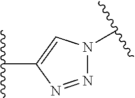 [SMILES: *c1cn(*)nn1]

The group L^{B}

In some embodiments, L^{B} is independently selected from ribofuranyl, glucopyranyl, galactopyranyl, mannopyranyl and allopyranyl.

In some embodiments, L^{B} is the following 6-membered monosaccharide moiety:

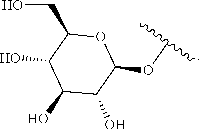 [SMILES: *O[C@@H]1O[C@H](CO)[C@@H](O)[C@H](O)[C@H]1O]

In some preferred embodiments, the saccharin compound is saccharin (2H-1λ⁶,2-benzothiazol-1,1,3-trione), according to Formula (IIIa):

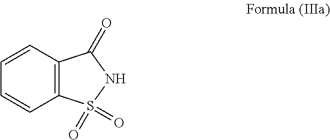 [SMILES: O=C1NS(=O)(=O)c2ccccc21]

In some embodiments, the saccharin compound is a saccharide salt according to Formula (IIIb):

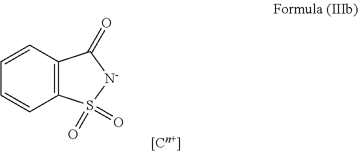 [SMILES: O=C1[N-]S(=O)(=O)c2ccccc21]

wherein [Cⁿ⁺] is a counter-cation selected from the group consisting of alkali metal ions, alkaline earth metal ions, transition metal ions, Al³⁺, ammonium or substituted ammonium ion and NO₂⁺, more preferably sodium or calcium ion.

In some embodiments, the saccharin compound is a compound according to Formula (IIIc):

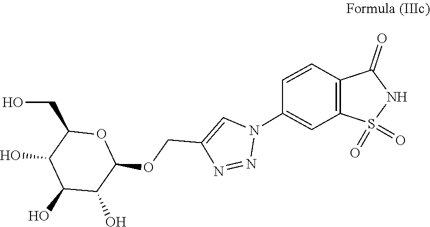 [SMILES: O=C1NS(=O)(=O)c2cc(-n3cc(CO[C@@H]4O[C@H](CO)[C@@H](O)[C@H](O)[C@H]4O)nn3)ccc21]

Any combination of salicylate compound, glycerol derivative and saccharin compound may be present, along with an optional flavouring agent, in the liquid compositions according to a first aspect of the invention.

In some embodiments, the glycerol derivative is a compound according to Formula (IIa), the saccharin compound is a compound according to Formula (IIIa) or (IIIb) and the salicylate compound is one or more salicylate compounds according to Formula (I).

In some embodiments, the glycerol derivative is a compound according to Formula (IIa), the saccharin compound is a compound according to Formula (IIIa) or (IIIb) and the salicylate compound is one or more salicylate compounds according to Formulae (Ia)-(Ie).

In some embodiments, the glycerol derivative is a compound according to Formula (IIa), the saccharin compound is a compound according to Formula (IIIa) or (IIIb) and the salicylate compound is a compound according to Formula (Ia).

### Isomers

Certain of the compounds described above may exist in one or more particular geometric, optical, enantiomeric, diastereoisomeric, epimeric, atropic, stereoisomeric, tautomeric, conformational, or anomeric forms, including but not limited to, cis- and trans-forms; E- and Z-forms; c-, t-, and r-forms; endo- and exo-forms; R-, S-, and meso-forms; D- and L-forms; d- and I-forms; (+) and (−) forms; keto-, enol-, and enolate forms; syn- and anti-forms; synclinal- and anticlinal-forms; α- and β-forms; axial and equatorial forms; boat-, chair-, twist-, envelope-, and halfchair-forms; and combinations thereof, hereinafter collectively referred to as “isomers” (or “isomeric forms”).

A reference to a class of structures may well include structurally isomeric forms falling within that class (e.g., C₁₋₇alkyl includes n-propyl and iso-propyl; butyl includes n-, iso-, sec-, and tert-butyl; methoxyphenyl includes ortho-, meta-, and para-methoxyphenyl).

However, reference to a specific group or substitution pattern is not intended to include other structural (or constitutional isomers) which differ with respect to the connections between atoms rather than by positions in space. For example, a reference to a methoxy group, —OCH₃, is not to be construed as a reference to its structural isomer, a hydroxymethyl group, —CH₂OH. Similarly, a reference to ortho-chlorophenyl is not to be construed as a reference to its structural isomer, meta-chlorophenyl.

The above exclusion does not pertain to tautomeric forms, for example, keto-, enol-, and enolate forms, as in, for example, the following tautomeric pairs: keto/enol (illustrated below), imine/enamine, amide/imino alcohol, amidine/amidine, nitroso/oxime, thioketone/enethiol, N nitroso/hydroxyazo, and nitro/aci-nitro.

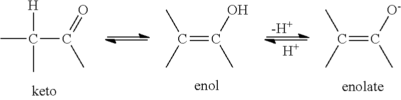

Note that specifically included in the term “isomer” are compounds with one or more isotopic substitutions. For example, H may be in any isotopic form, including ¹H, ²H (D), and ³H (T); C may be in any isotopic form, including ¹²C, ¹³C, and ¹⁴C; O may be in any isotopic form, including ¹⁶O and ¹⁸O; and the like.

Unless otherwise specified, a reference to a particular compound includes all such isomeric forms, including mixtures (e.g., racemic mixtures) thereof. Methods for the preparation (e.g., asymmetric synthesis) and separation (e.g., fractional crystallisation and chromatographic means) of such isomeric forms are known in the art or are readily obtained by adapting known methods in a known manner.

### Salts

As explained above, the salicylate compound and/or the saccharin compound can be provided as salts. As such, in this specification the terms “salicylate compound” and “saccharin compound” include salts thereof.

It may be convenient or desirable to prepare, purify, and/or handle a corresponding salt of a compound, for example, a pharmaceutically-acceptable salt. Examples of pharmaceutically acceptable salts are discussed in Berge et al., 1977, “Pharmaceutically Acceptable Salts,” J. Pharm. Sci., Vol. 66, pp. 1-19.

For example, when the salicylate compound or saccharin compound exists as an anion, or has a functional group which may be anionic (e.g., —COOH may be —COO⁻), then a salt may be formed with a suitable cation (such as [Cⁿ⁺+] described above). Examples of suitable inorganic cations include, but are not limited to, alkali metal ions such as Na⁺ and K⁺, alkaline earth cations such as Ca²⁺ and Mg²⁺, and other cations such as Al³⁺. Examples of suitable organic cations include, but are not limited to, ammonium ion (i.e., NH⁴⁺) and substituted ammonium ions (e.g., NH₃R⁺, NH₂R²⁺, NHR³⁺, NR⁴⁺). Examples of some suitable substituted ammonium ions are those derived from: ethylamine, diethylamine, dicyclohexylamine, triethylamine, butylamine, ethylenediamine, ethanolamine, diethanolamine, piperazine, benzylamine, phenylbenzylamine, choline, meglumine, and tromethamine, as well as amino acids, such as lysine and arginine. An example of a common quaternary ammonium ion is N(CH₃)⁴⁺.

If a compound is cationic, or has a functional group which may be cationic (e.g., —NH₂ may be —NH₃⁺), then a salt may be formed with a suitable anion. Examples of suitable inorganic anions include, but are not limited to, those derived from the following inorganic acids: hydrochloric, hydrobromic, hydroiodic, sulfuric, sulfurous, nitric, nitrous, phosphoric, and phosphorous. Examples of suitable organic anions include, but are not limited to, those derived from the following organic acids: 2-acetyoxybenzoic, acetic, ascorbic, aspartic, benzoic, camphorsulfonic, cinnamic, citric, edetic, ethanedisulfonic, ethanesulfonic, fumaric, glucheptonic, gluconic, glutamic, glycolic, hydroxymaleic, hydroxynaphthalene carboxylic, isethionic, lactic, lactobionic, lauric, maleic, malic, methanesulfonic, mucic, oleic, oxalic, palmitic, pamoic, pantothenic, phenylacetic, phenylsulfonic, propionic, pyruvic, salicylic, stearic, succinic, sulfanilic, tartaric, toluenesulfonic, and valeric. Examples of suitable polymeric organic anions include, but are not limited to, those derived from the following polymeric acids: tannic acid, carboxymethyl cellulose.

Unless otherwise specified, a reference to a particular compound also includes salt forms thereof.

### Solvates and Hydrates

It may be convenient or desirable to prepare, purify, and/or handle a corresponding solvate of a compound. The term “solvate” is used herein in the conventional sense to refer to a complex of solute (e.g., compound, salt of compound) and solvent. If the solvent is water, the solvate may be conveniently referred to as a hydrate, for example, a mono-hydrate, a di hydrate, a tri-hydrate, etc.

Unless otherwise specified, a reference to a particular compound also includes solvate and hydrate forms thereof.

### Prodrugs

It may be convenient or desirable to prepare, purify, and/or handle a compound, especially the salicylate compound, in the form of a prodrug. The term “prodrug,” as used herein, pertains to a compound which, when metabolised (e.g., in vivo), yields the desired active compound. Typically, the prodrug is inactive, or less active than the desired active compound, but may provide advantageous handling, administration, or metabolic properties.

In one embodiment, the glycerin triacetate used herein may suitably comprise in the range from 0 to 1 wt %, preferably 0 to 0.5 wt %, more preferably 0 to 0.1 wt %, particularly 0 to 0.05 wt %, and especially 0 to 0.015% of glycerin monoacetate and/or glycerine diacetate.

The glycerin triacetate may also suitably comprise in the range from 0 to 100 ppm, preferably 0 to 20 ppm, more preferably 0 to 10 ppm, particularly 0 to 2 ppm, and especially 0 to 1 ppm of glycerine monoacetate and/or glycerine diacetate.

In one embodiment, the glycerin triacetate may suitably comprise in the range from 0 to 50 ppm, preferably 0 to 10 ppm, more preferably 0 to 5 ppm, particularly 0 to 1 ppm, and especially 0 to 0.5 ppm of glycerine.

The glycerin triacetate suitably comprises in the range from 0 to 100 ppm, preferably 0 to 20 ppm, more preferably 0 to 10 ppm, particularly 0 to 2 ppm, and particularly 0 to 1 ppm of non-polar materials, such as colour pigments and/or soaps.

In one preferred embodiment, the glycerin triacetate is substantially anhydrous, suitably comprising in the range from 0 to 0.5 wt %, preferably less than 0.3 wt %, more preferably less than 0.2 wt %, particularly less than 0.1 wt %, and especially less than 0.05 wt % of water.

A reduction in water content of the glycerin triacetate ensures a low water content in the composition. This provides increased stability of the liquid composition because salicylate compounds such as aspirin are vulnerable to hydrolysis in the presence of water to produce salicylic acid and other acids as by-products. If an aspirin composition contains 10 wt % salicylic acid with reference to the total amount of aspirin and salicylic acid present, the composition is no longer deemed to be pharmaceutically acceptable. Thus an increase in aspirin stability results in an extended shelf-life of the pharmaceutical composition.

Preferably the composition comprises at least 5 wt % glycerin triacetate, for example at least 10 wt %, at least 15 wt %, at least 20 wt % or at least 25 wt %. Most preferably, the composition comprises at least 90 wt % glycerin triacetate, for example at least 91 wt %, at least 92 wt %, at least 93 wt %, at least 94 wt %, at least 95 wt % or at least 96 wt %.

Preferably the composition comprises up to 99.9 wt % glycerin triacetate, for example up to 99 wt %, up to 98 wt %, up to 97 wt % or up to 96.5 wt %.

The concentration of the glycerin triacetate in the composition according to the invention is suitably in the range from 90 to 99%, preferably 94 to 98%, more preferably 95 to 97.5%, particularly 95.5 to 97%, and especially 96 to 96.5% by weight based on the total weight of the composition.

At this concentration of glycerin triacetate excellent solubilisation and stability of the salicylate compound is observed and a shelf-life of over 1 year, preferably about 18 months or 2 years, may be achieved. As used herein, “stability” refers to the resistance to degradation of the salicylate compound. Higher stability means that after a given period of time lower levels of contaminants will accumulate in the composition. These contaminants may be the products of the hydrolysis of the salicylate compound. The stability of a composition may be measured using the method described below.

Preferably the composition comprises at least 0.1 wt % salicylate compound, e.g. aspirin, for example at least 0.125 wt %, 0.2 wt %, 0.25 wt %, 0.5 wt %, at least 1 wt %, at least 1.5 wt %, at least 2.0 wt % or at least 2.5 wt %. In this way a pharmaceutically effective amount of salicylate compound, e.g. aspirin may be provided in a small amount of liquid composition.

Preferably the composition comprises up to 10 wt % salicylate compound, e.g. aspirin, for example up to 9 wt %, up to 8 wt %, up to 7 wt %, up to 6 wt %, up to 5 wt %, up to 4 wt %, up to 3 wt %, up to 2.5 wt %, up to 2 wt %, up to 1 wt %, up to 0.5 wt %, up to 0.25 wt %, up to 0.2 wt %, up to 0.15 wt % or up to 0.125 wt %. In this way the stability and solubility of the salicylate compound, e.g. aspirin, in the composition is maximised.

When the concentration of salicylate compound, e.g. aspirin, exceeds this amount there is an increased risk that not all the aspirin in the liquid composition will be fully solubilised, which may lead to a granular precipitate having the disadvantages associated with solid aspirin compositions.

In some embodiments, the concentration of salicylate compound in the composition according to the invention is suitably in the range from 0.05 to 7%, preferably 0.1% to 5%, more preferably 0.12% to 2.6% by weight based on the total weight of the composition.

In some embodiments, the concentration of the salicylate compound, e.g. aspirin, in the composition according to the invention is suitably in the range from 0.5 to 7%, preferably 1 to 5%, more preferably 2 to 3%, particularly 2.3 to 2.7%, and especially 2.4 to 2.6% by weight based on the total weight of the composition. In some embodiments, the concentration of the salicylate compound, e.g. aspirin, in the composition according to the invention is 2.5 wt %.

In some embodiments, the concentration of salicylate compound in the composition according to the invention is suitably in the range from 0.05 to 0.5%, preferably 0.1 to 0.4%, more preferably 0.2 to 0.3%, particularly 0.22 to 0.28%, and especially 0.24 to 0.26% by weight based on the total weight of the composition. In some embodiments, the concentration of the salicylate compound, e.g. aspirin, in the composition according to the invention is 0.25 wt %.

In some embodiments, the concentration of salicylate compound in the composition according to the invention is suitably in the range from 0.05 to 0.5%, preferably 0.07 to 0.2%, more preferably 0.1 to 0.15%, particularly 0.11 to 0.14%, and especially 0.12 to 0.13% by weight based on the total weight of the composition. In some embodiments, the concentration of the salicylate compound, e.g. aspirin, in the composition according to the invention is 0.125 wt %.

In one preferred embodiment, the salicylate compound is substantially anhydrous, suitably comprising in the range from 0 to 0.5 wt %, preferably less than 0.3 wt %, more preferably less than 0.2 wt %, particularly less than 0.1 wt %, and especially less than 0.05 wt % of water.

The composition may comprise at least 0.1 wt % saccharin, for example at least 0.2 wt %, at least 0.3 wt %, at least 0.4 wt %, at least 0.5 wt %, at least 0.6 wt %, at least 0.7 wt %, at least 0.8 wt % or at least 0.9 wt %. In this way, excellent stability and solubilisation of the salicylate compound is achieved.

The composition may comprise up to 5 wt % saccharin, for example up to 4 wt %, up to 3 wt %, up to 2 wt %, up to 1.5 wt %, up to 1.2 wt % or up to 1.1 wt %.

The concentration of the saccharin in the composition according to the invention is suitably in the range from 0.1 to 4%, preferably 0.4 to 3%, more preferably 0.7 to 1.5%, particularly 0.8 to 1.2%, and especially 0.9 to 1.1% by weight based on the total weight of the composition.

In one preferred embodiment, the saccharin compound is substantially anhydrous, suitably comprising in the range from 0 to 0.5 wt %, preferably less than 0.3 wt %, more preferably less than 0.2 wt %, particularly less than 0.1 wt %, and especially less than 0.05 wt % of water.

The composition according to the invention is preferably substantially anhydrous, suitably comprising in the range from 0 to 0.5 wt %, preferably less than 0.3 wt %, more preferably less than 0.2 wt %, particularly less than 0.1 wt %, and especially less than 0.05 wt % of water.

The composition according to the invention preferably comprises less than 0.5 wt % water, for example less than 0.4 wt %, less than 0.3 wt %, less than 0.2 wt %, or less than 0.1 wt % water. In some embodiments the composition according to the invention comprises less than 1000 ppm water, for example less than 900 ppm, less than 800 ppm, less than 700 ppm, less than 600 ppm or less than 500 ppm water.

As explained above, keeping the level of water in the composition low increases the stability of the salicylate compound in the composition thereby increasing the shelf-life.

The liquid composition of the invention may optionally also comprise one or more other pharmaceutically acceptable ingredients well known to those skilled in the art, including, but not limited to, pharmaceutically acceptable carriers, diluents, excipients, adjuvants, fillers, buffers, preservatives, anti-oxidants, lubricants, stabilisers, solubilisers, surfactants (e.g., wetting agents), masking agents, colouring agents, flavouring agents, and sweetening agents. The formulation may further comprise other active agents, for example, other therapeutic or prophylactic agents.

The term “pharmaceutically acceptable,” as used herein, pertains to compounds, ingredients, materials, compositions, dosage forms, etc., which are, within the scope of sound medical judgment, suitable for use in contact with the tissues of the subject in question (e.g., human) without excessive toxicity, irritation, allergic response, or other problem or complication, commensurate with a reasonable benefit/risk ratio.

Each carrier, diluent, excipient, etc. must also be “acceptable” in the sense of being compatible with the other ingredients of the formulation without exerting a detrimental effect on the solubility or stability of the salicylate compound.

Suitable carriers, diluents, excipients, etc. can be found in standard pharmaceutical texts, for example, Reminaton's Pharmaceutical Sciences, 18th edition, Mack Publishing Company, Easton, Pa., 1990; and Handbook of Pharmaceutical Excipients, 5th edition, 2005.

The composition may also comprise an optional masking or flavouring agent. Suitable masking agents may mask or hide any objectionable flavour of the base composition. The flavouring agent preferably comprises any natural or synthetically prepared fruit or botanical flavouring agent, or mixtures thereof. Suitable natural or artificial fruit flavouring agents include lemon, orange, grapefruit, strawberry, banana, pear, kiwi, grape, apple, mango, pineapple, passion fruit, raspberry, and mixtures thereof. The fruit flavouring agent is suitably in anhydrous form such as dried juice or oil, preferably oil. Suitable botanical flavours include any member of the mint family such as spearmint, peppermint, watermint, apple mint; and other flavours such as Jamaica, marigold, chrysanthemum, tea, chamomile, ginger, valerian, yohimbe, hops, eriodictyon, ginseng, bilberry, rice, red wine, mango, peony, lemon balm, nut gall, oak chip, lavender, walnut, gentiam, luo han guo, cinnamon, angelica, aloe, agrimony, yarrow, and mixtures thereof. The botanical flavouring agent is suitably an anhydrous concentrate or an extract, for example in the form of oil or dried to form a powder.

The flavouring agent preferably comprises, consists essentially of, or consists of, mint flavouring agent, more preferably in the form of oil, particularly spearmint and/or peppermint oil, and especially spearmint oil.

The composition may comprise at least 0 wt %, for example at least 0.05 wt %, at least 0.06 wt %, at least 0.07 wt %, at least 0.08 wt %, at least 0.09 wt % or at least 0.1 wt % flavouring agent, preferably oil, more preferably mint oil.

In this way, an additional stability enhancement of the salicylate compound may be observed.

The composition may comprise up to 3 wt %, for example up to 2.5 wt %, up to 2 wt %, up to 1.5 wt %, up to 1 wt %, up to 0.5 wt %, up to 0.2 wt %, up to 0.18 wt % or up to 0.16 wt % flavouring agent, preferably oil, more preferably mint oil.

The concentration of flavouring agent, preferably oil, in the composition is suitably in the range from 0 to 3%, preferably 0.05 to 1%, more preferably 0.1 to 0.2%, particularly 0.12 to 0.18%, and especially 0.14 to 0.16% by weight based on the total weight of the composition.

The composition may also comprise an optional, additional natural or artificial sweetener or sweetening agent (in addition to saccharin). Suitable sweeteners are natural sugars which may be granulated or powdered, and include sucrose, fructose, dextrose, maltose, lactose, xylitol, polyols, and mixtures thereof.

In other embodiments, artificial sweeteners may be utilized in the composition. Suitable optional artificial sweeteners (in addition to saccharin) include, for example, cyclamates, sucralose, acesulfam-K, L-aspartyl-L-phenylalanine lower alkyl ester sweeteners (e.g. aspartame), L-aspartyl 1-D -alanine amides, L-aspartyl-D-serine amides, L-aspartyl-L-1-hydroxymethylalkaneamides, L-aspartyl-1-hydroxyethyalkaneamides, L-aspartyl-D-phenylglycine esters and amides.

The concentration of the optional, preferably artificial, sweetener (not including saccharin) in the composition is suitably in the range from 0 to 5%, preferably 0.4 to 2%, more preferably 0.7 to 1.3%, particularly 0.8 to 1.2%, and especially 0.9 to 1.1% by weight based on the total weight of the composition.

The composition according to the present invention may also contain an antioxidant. Suitable examples of antioxidants include a phenolic compound, a plant extract, or a sulphur-containing compound. The antioxidant may be ascorbic acid or a salt thereof, vitamin E, CoQIO, tocopherols, lipid soluble derivatives of more polar antioxidants such as ascorbyl fatty acid esters (e.g. ascorbyl palmitate), plant extracts (e.g. rosemary, sage and oregano oils, green tea extract), algal extracts, and synthetic antioxidants (e.g., butylated hydroxytoluene (BHT), tert-butylhydroquinone (TBHQ), butylated hydroxyanisole (BHA), ethoxyquin, alkyl gallates, hydroquinones, tocotrienols), and combinations thereof.

The concentration of the antioxidant in the composition is preferably in the range from 0 to 2%, more preferably 0.01 to 1.5%, particularly 0.1 to 1%, and especially 0.15 to 0.5% by weight based on the total weight of the composition.

The composition according to one embodiment of the invention comprises, consists essentially of, or consists of, aspirin, glycerin triacetate, saccharin and an optional flavouring agent.

The aspirin is preferably completely soluble in the composition and the composition is more preferably free of particulate material, as described above. The composition exhibits surprisingly improved stability to degradation. We have discovered that the flavouring agent, particularly mint oil, can also surprisingly contribute to the improved stability to degradation.

The composition according to the present invention is preferably stable, measured as described herein, suitably having a salicylate compound, e.g. aspirin degradation rate at 25° C. of less than 0.05%/day, preferably less than 0.04%/day, more preferably less than 0.03%/day, particularly less than 0.02%/day, and especially less than 0.015%/day.

The composition suitably has a salicylate compounds, e.g. aspirin degradation rate in mg per gram of composition per day at 25° C., measured as described herein, of less than 0.01 mg/g/day, preferably less than 0.007 mg/g/day, more preferably less than 0.006 mg/g/day, particularly less than 0.005 mg/g/day, and especially less than 0.004 mg/g/day.

The liquid composition described herein is particularly suitable for oral use as an alternative to normal aspirin tablets. One particular use is to orally administer to stroke or heart attack victims immediately after the attack, e.g. suitably within 12 hours, preferably within 8 hours, more preferably within 4 hours, particularly within 2 hours, and especially within 1 hour of the heart attack, e.g. in the ambulance on the way to hospital.

### Formulations

The liquid composition according to the present disclosure may sometimes be referred to as ‘enhanced liquid aspirin’ or ‘ELA’.

Liquid compositions according to the present invention may be formulated as a pharmaceutical composition or medicament.

Thus, in some embodiment the liquid composition is a pharmaceutical composition or medicament. Methods of making a pharmaceutical composition or medicament comprising admixing the salicylate compound, glycerol derivative and saccharin compound are provided, which methods may optionally further comprise admixing together with one or more other pharmaceutically acceptable ingredients well known to those skilled in the art, e.g., carriers, adjuvants, excipients, etc. If formulated as discrete units (e.g., tablets/capsules, etc.), each unit contains a predetermined amount (dosage) of the active compound(s), i.e. the salicylate compound and/or glycerol derivative and/or saccharin compound.

The term “pharmaceutically acceptable” as used herein pertains to compounds, ingredients, materials, compositions, dosage forms, etc., which are, within the scope of sound medical/veterinary judgment, suitable for use in contact with the tissues of the subject in question without excessive toxicity, irritation, allergic response, or other problem or complication, commensurate with a reasonable benefit/risk ratio.

Each carrier, adjuvant, excipient, etc. must also be “acceptable” in the sense of being compatible with the other ingredients of the formulation.

Suitable carriers, adjuvants, excipients, etc. can be found in standard pharmaceutical texts, for example, Remington's Pharmaceutical Sciences, 18th edition, Mack Publishing Company, Easton, Pa., 1990; and Handbook of Pharmaceutical Excipients, 2nd edition, 1994.

Pharmaceutical formulations may be prepared by any methods well known in the art of pharmacy. Such methods include the step of bringing into association the active compound with a carrier which constitutes one or more accessory ingredients. In general, the formulations are prepared by uniformly and intimately bringing into association the active compound with carriers (e.g., liquid carriers, finely divided solid carrier, etc.), and then shaping the product, if necessary.

The formulation may be prepared to provide for rapid or slow release; immediate, delayed, timed, or sustained release; or a combination thereof.

Formulations may suitably be in the form of liquids, elixirs, syrups, mouthwashes, drops, capsules (including, e.g., gel capsules, hard and soft gelatin capsules), ampoules, sprays, mists, aerosols or vapours. Liquid formulations may be suitable for oral administration, e.g. in the form of a gel capsule filled with the liquid composition, or for intravenous or intraarterial administration, e.g. by injection. Liquid formulations may also be suitable for inhalation through the nose or mouth, e.g. when delivered as an aerosol or vapour.

Formulations suitable for inhalation administration, include, for example, nasal spray, nasal drops, or by aerosol administration by nebuliser. Formulations suitable for pulmonary administration (e.g., by inhalation or insufflation therapy) include those presented as an aerosol spray from a pressurised pack, with the use of a suitable propellant, such as dichlorodifluoromethane, trichlorofluoromethane, dichoro-tetrafluoroethane, carbon dioxide, or other suitable gases.

Formulations may suitably be provided as a patch, adhesive plaster, bandage, dressing, or the like which is impregnated with the liquid composition and optionally one or more other pharmaceutically acceptable ingredients, including, for example, penetration, permeation, and absorption enhancers.

### Uses

The present invention provides the use of a composition according to the present disclosure to enhance productivity of an animal. Also provided is a method of enhancing productivity of an animal, the method comprising administering to the animal an effective amount of a composition according to the present disclosure.

In some embodiments, the present invention provides the non-therapeutic use of a composition according to the present disclosure to enhance productivity of an animal. Also provided is a non-therapeutic method of enhancing productivity of an animal, the method comprising administering to the animal an effective amount of a composition according to the present disclosure.

As used herein, a ‘non-therapeutic’ method of the present invention refers to the use of a composition according to the present disclosure for non-therapeutic purposes, for example enhancing weight gain or egg production in a healthy animal (e.g. an animal not diagnosed with or suffering from any disease or condition, e.g. an animal not diagnosed with or suffering from any disease or condition known to be treatable with salicylate compounds). Non-therapeutic methods of the present invention do not encompass the use of a composition according to the present invention for the treatment and/or prevention of any disease or condition. For example, non-therapeutic methods of the present invention do not encompass the use or effect of a composition according to the present invention in the treatment or prevention of a disease or condition, for example cancer, cardiovascular disease, inflammation, or pain. Non-therapeutic methods of the invention do not include and/or encompass methods for treatment of the human or animal body by surgery or therapy and diagnostic methods practised on the human or animal body.

As used herein, ‘productivity’ of an animal refers to the efficiency in production of animal derived products (such as milk, meat or eggs). ‘Productivity’ may refer to any production trait of an animal, including growth rate, weight gain, egg production (including egg laying rate and egg size), and milk production.

Enhancing productivity of an animal refers to enhancing (i.e. promoting, facilitating) an increase in one or more production traits of an animal, as described herein, as compared to an appropriate control condition. Appropriate control conditions may include the same animal prior to administration of the composition, or an equivalent animal, observed at the same time point, not administered with the composition.

An ‘effective amount’ of a composition according to the present disclosure is an amount sufficient to show the claimed effect in an animal (e.g. enhanced productivity, e.g. enhanced weight gain).

It will be appreciated that a method (e.g. a non-therapeutic method) described herein may enhance more than one of the production traits described herein. A given method may be evaluated for efficacy in enhancing production traits described herein using suitable assays. For example, the assays may be e.g. in vitro assays, optionally cell-based assays or cell-free assays. In some embodiments, the assays may be e.g. ex vivo assays, i.e. performed using cells/tissue/an organ obtained from a subject. In some embodiments, the assays may be e.g. in vivo assays, i.e. performed in non-human animals (e.g. live non-human animals).

In some embodiments, the present invention provides a method of enhancing the growth rate of an animal, the method comprising administering to the animal an effective amount of a composition according to the present disclosure. In some embodiments, the present invention provides a non-therapeutic method of enhancing the growth rate of an animal, the method comprising administering to the animal an effective amount of a composition according to the present disclosure. Animal growth rates may be determined by measurement of a number of parameters including body weight (e.g. live body weight), linear body measurements, animal girth and intestinal villus height. Live body weight may be measured using traditional scales or estimated from body measurements. Methods of measuring animal growth are described, for example, Wangchuck et al. Journal of Applied Animal Research. (2018). 46(1):349-352; or Example 7 of the present application.

In some embodiments, evaluation of animal growth is performed after more than 3 days, e.g. one of ≥5 days, ≥10 days, ≥15 days, ≥20 days, ≥25 days, ≥30 days, ≥35 days, ≥40 days, ≥35 days, ≥50 days, ≥55 days, ≥60 days, ≥65 days, ≥70 days, ≥75 days, ≥80 days, ≥85 days, ≥90 days, ≥95 days or 100 days following administration of the first quantity of the composition.

In some embodiments, administration to an animal of a composition according to the present disclosure enhances animal growth (e.g. expressed as intestinal villus height) of greater than 1 times, e.g. one of ≥1.01 times, ≥1.02 times, ≥1.03 times, ≥1.04 times, ≥1.05 times, ≥1.06 times, ≥1.07 times, ≥1.08 times, ≥1.09 times, ≥1.10 times, ≥1.11 times, ≥1.12 times, ≥1.13 times, ≥1.14 times, ≥1.15 times, ≥1.16 times, ≥1.17 times, ≥1.18 times, ≥1.19 times, 1.20 times, ≥1.21 times, ≥1.22 times, ≥1.23 times, ≥1.24 times, 1.25 times, ≥1.26 times, ≥1.27 times, 1.28 times, ≥1.29 times, ≥1.3 times, ≥1.4 times, ≥1.5 times, ≥1.6 times, ≥1.7 times, ≥1.8 times, ≥1.9 times, ≥2 times, ≥3 times, ≥4 times, ≥5 times the growth observed at the same timepoint in an equivalent animal, in the absence of administration of the composition (or following administration with an appropriate control composition known not to influence growth rate).

In some embodiments, the present invention provides a method of enhancing (e.g. promoting or facilitating) weight gain (e.g. live body weight gain) in an animal, the method comprising administering to the animal an effective amount of a composition according to the present disclosure. In some embodiments, the present invention provides a non-therapeutic method of enhancing (e.g. promoting or facilitating) weight gain (e.g. live body weight gain) in an animal, the method comprising administering to the animal an effective amount of a composition according to the present disclosure.

Weight gain may be expressed as % weight change over a given time period following administration of the composition, relative to the weight prior to administration of the composition. In some embodiments, enhancement of weight gain is assessed by comparing the % weight change of an animal having been administered a composition according to the present disclosure to the % weight change of an equivalent animal observed at the same time point, in the absence of administration of the composition (or following administration with an appropriate control composition known not to influence body weight). In some embodiments, evaluation of weight gain for the purposes of such comparison is performed after more than 3 days, e.g. one of ≥5 days, ≥10 days, ≥15 days, ≥20 days, ≥25 days, ≥30 days, ≥35 days, ≥40 days, ≥45 days, ≥50 days, ≥55 days, ≥60 days, ≥65 days, ≥70 days, ≥75 days, ≥80 days, ≥85 days, ≥90 days, ≥95 days or ≥100 days following administration of the first quantity of the composition.

Enhancing (e.g. promoting or facilitating) weight gain in an animal may refer to increasing the weight of an animal or may refer to reducing (e.g. inhibiting, slowing or preventing) weight loss in an animal. For example, the use of a composition according to the present disclosure to enhance weight gain in an animal may refer to the use of a composition according to the present disclosure to reduce weight loss in an animal (e.g. as compared to weight loss observed at the same time point in an equivalent animal not administered with the composition, or administered with an appropriate control composition known not to influence body weight). That is, in some embodiments, the present invention provides a method of enhancing weight gain or reducing weight loss in an animal, the method comprising administering to the animal an effective amount of a composition according to the present disclosure. In some embodiments, the present invention provides a non-therapeutic method of enhancing weight gain or reducing weight loss in an animal, the method comprising administering to the animal an effective amount of a composition according to the present disclosure.

In some embodiments, administration to an animal of a composition according to the present disclosure facilitates weight gain (e.g. expressed as % weight gain) of greater than 1 times, e.g. one of ≥1.01 times, ≥1.02 times, ≥1.03 times, ≥1.04 times, ≥1.05 times, ≥1.1 times, ≥1.2 times, ≥1.3 times, ≥1.4 times, ≥1.5 times, ≥1.6 times, ≥1.7 times, ≥1.8 times, ≥1.9 times, ≥2 times, ≥3 times, ≥4 times, ≥5 times the level of weight gain observed at the same timepoint in an equivalent animal, in the absence of administration of the composition (or following administration with an appropriate control composition known not to influence body weight).

In some embodiments, administration to an animal of a composition according to the present disclosure facilitates weight gain (e.g. expressed as % weight gain) of greater than 1 times e.g. one of ≥1.01 times, ≥1.02 times, ≥1.03 times, ≥1.04 times, ≥1.05 times, ≥1.1 times, ≥1.2 times, ≥1.3 times, ≥1.4 times, ≥1.5 times, ≥1.6 times, ≥1.7 times, ≥1.8 times, ≥1.9 times, ≥2 times, ≥3 times, ≥4 times, ≥5 times the level of weight gain of an equivalent animal receiving the same diet in the absence of administration of the composition (or following administration with an appropriate control composition known not to influence body weight) observed after 3 days, e.g. one of ≥5 days, ≥10 days, ≥15 days, ≥20 days, ≥25 days, ≥30 days, ≥35 days, ≥40 days, ≥35 days, ≥50 days, ≥55 days, ≥60 days, ≥65 days, ≥70 days, ≥75 days, ≥80 days, ≥85 days, ≥90 days, ≥95 days or ≥100 days following administration of the first quantity of the composition.

In some embodiments, administration to an animal of a composition according to the present disclosure facilitates weight gain of an animal of approximately 5% more than an equivalent animal receiving the same diet in the absence of administration of the composition (or following administration with an appropriate control composition known not to influence body weight), that is one of at least 6%, 7%, 8%, 9%, 10%, 15%, 20%, 30%, 40%, 50%, 60%, 70%, 80%, 90%, 100%, 150% or 200% more weight gain, after a period of at least 3 days, that is one ≥5 days, ≥10 days, ≥15 days, ≥20 days, ≥25 days, ≥30 days, ≥35 days, ≥40 days, ≥45 days, ≥50 days, ≥55 days, ≥60 days, ≥65 days, ≥70 days, ≥75 days, ≥80 days, ≥85 days, ≥90 days, ≥95 days or ≥100 days following administration of the first quantity of the composition.

In some embodiments, the present invention provides a method of enhancing egg production in an animal, the method comprising administering to the animal an effective amount of a composition according to the present disclosure. In some embodiments, the present invention provides a non-therapeutic method of enhancing egg production in an animal, the method comprising administering to the animal an effective amount of a composition according to the present disclosure. Egg production may be assessed by measuring traits including egg laying rate, egg size, egg weight, shell strength, shell thickness, albumen quality (e.g. Haugh unit) and yolk weight. Egg quality assessments are described, for example, in Bagheri et al. Anim Nutr. (2018). 5(2):130-133.

Egg production may be expressed as egg laying rate (e.g. egg number per animal per day), over a given time period following administration of the composition. In some embodiments, enhancement of egg production is assessed by comparing the egg laying rate of an animal having been administered a composition according to the present disclosure to the egg laying rate of an equivalent animal observed at the same time point, in the absence of administration of the composition (or following administration with an appropriate control composition known not to influence egg production). In some embodiments, evaluation of egg production for the purposes of such comparison is performed after more than 7 days, e.g. one of ≥10 days, ≥15 days, ≥20 days, ≥25 days, ≥30 days, ≥35 days, ≥40 days, ≥35 days, ≥50 days, ≥55 days, ≥60 days, ≥65 days, ≥70 days, ≥75 days, ≥80 days, ≥85 days, ≥90 days, ≥95 days or ≥100 days following administration of the first quantity of the composition.

Egg production may be expressed as egg size (e.g. average egg weight), of eggs produced over a given time period following administration of the composition. In some embodiments, enhancement of egg production is assessed by comparing the average egg weight of eggs produced by an animal having been administered a composition according to the present disclosure to the average egg weight of an equivalent animal observed at the same time point, in the absence of administration of the composition (or following administration with an appropriate control composition known not to influence egg production). In some embodiments, evaluation of egg production for the purposes of such comparison is performed after more than 7 days, e.g. one of ≥10 days, ≥15 days, ≥20 days, ≥25 days, ≥30 days, ≥35 days, ≥40 days, ≥35 days, ≥50 days, ≥55 days, ≥60 days, ≥65 days, ≥70 days, ≥75 days, ≥80 days, ≥85 days, ≥90 days, ≥95 days or ≥100 days following administration of the first quantity of the composition.

In some embodiments, administration to an animal of a composition according to the present disclosure enhances egg production (e.g. expressed as average egg weight) of greater than 1 times, e.g. one of ≥1.01 times, ≥1.02 times, ≥1.03 times, ≥1.04 times, ≥1.05 times, ≥1.1 times, ≥1.2 times, ≥1.3 times, ≥1.4 times, ≥1.5 times, ≥1.6 times, ≥1.7 times, ≥1.8 times, ≥1.9 times, ≥2 times, ≥3 times, ≥4 times, ≥5 times the level of egg production observed at the same timepoint in an equivalent animal, in the absence of administration of the composition (or following administration with an appropriate control composition known not to influence egg production).

In some embodiments, the present invention provides a method of enhancing milk production in an animal, the method comprising administering to the animal an effective amount of a composition according to the present disclosure. In some embodiments, the present invention provides a non-therapeutic method of enhancing milk production in an animal, the method comprising administering to the animal an effective amount of a composition according to the present disclosure. Milk production may be assessed by measuring traits including milk yield, udder conformation (including udder height, udder size, and teat characteristics), milk composition (e.g. % fat and % protein) and feed efficiency (e.g. kg of milk per kg of dry matter intake). Methods for determining milk production capacity are described, for example, in Saleh et al. Sci Rep. (2023) 13(1):16193; Golovan et al. E3S Web Conf. (2020) 175:03001 and Tassoul et al. Journal of Dairy Science. (2009). 92(4):1734-1740.

Milk production may be expressed as kg milk yield over a given time period following administration of the composition. In some embodiments, enhancement of milk production is assessed by comparing the milk yield of an animal having been administered a composition according to the present disclosure to the milk yield of an equivalent animal observed at the same time point, in the absence of administration of the composition (or following administration with an appropriate control composition known not to influence milk production). In some embodiments, evaluation of milk production for the purposes of such comparison is performed after more than 7 days, e.g. one of ≥10 days, ≥15 days, ≥20 days, ≥25 days, ≥30 days, ≥35 days, ≥40 days, ≥35 days, ≥50 days, ≥55 days, ≥60 days, ≥65 days, ≥70 days, ≥75 days, ≥80 days, ≥85 days, ≥90 days, ≥95 days or ≥100 days following administration of the first quantity of the composition.

Enhancing milk production in an animal may refer to increasing the milk production of an animal or may refer to reducing (e.g. inhibiting, slowing or preventing) a decline in milk production of an animal (e.g. in later stages of lactation). For example, the use of a composition according to the present disclosure to enhance milk production in an animal may refer to the use of a composition according to the present disclosure to reduce the decline in milk production of an animal (e.g. as observed in later stages of lactation, e.g. as compared to the decline in milk production observed at the same time point in an equivalent animal not administered with the composition, or administered with an appropriate control composition known not to influence milk production).

In some embodiments, administration to an animal of a composition according to the present disclosure enhances milk production (e.g. expressed as milk yield) of greater than 1 times, e.g. one of ≥1.01 times, ≥1.02 times, ≥1.03 times, ≥1.04 times, ≥1.05 times, ≥1.1 times, ≥1.2 times, ≥1.3 times, ≥1.4 times, ≥1.5 times, ≥1.6 times, ≥1.7 times, ≥1.8 times, ≥1.9 times, ≥2 times, ≥3 times, ≥4 times, ≥5 times the level of milk production observed at the same timepoint in an equivalent animal, in the absence of administration of the composition (or following administration with an appropriate control composition known not to influence milk production).

The compositions of the present disclosure may be used as a feed additive, or diet supplement for a subject described herein.

### Subjects

A subject according to the present invention may be any animal, preferably any non-human animal. Animals (e.g. non-human animals) according to the present invention may be poultry (including chickens, turkey, ducks, geese, and pheasants), pigs, cattle (including cows, e.g. dairy cows, or any animal in the order Bos), goats, sheep, horses (including any animal in the order Equidae), deer, rabbits, fish and companion animals (including cats, dogs, rodents (including any animal in the order Rodentia)).

In some embodiments, the subject is a livestock animal. Livestock animals refer to domesticated animals raised to generate products for consumption (such as meat, milk and eggs). Livestock animals may include poultry (including chickens (e.g. hens), turkey, ducks, geese, and pheasants), pigs, cattle, goats and sheep. In some embodiments, subjects may be poultry, cattle, sheep or pigs.

In some embodiments, the subject is a ruminant animal. Ruminant animals include cattle, sheep and goats. In some embodiments, the subject is a monogastric animal. Monogastric animals include pigs, poultry (including chickens (e.g. hens), turkey, ducks, geese, and pheasants), horses, dogs and cats.

In some embodiments, the subject is a ruminant livestock animal. In some embodiments, the subject is a monogastric livestock animal.

In some embodiments, the subject is a mammal or a bird. In some embodiments, the subject is a bird (e.g. chicken (including any animal in the order Galliformes) and ducks (including any animal in the order Anseriformes). In some embodiments, the subject is a chicken. In some embodiments the subject is a mammal (e.g. rabbit, guinea pig, rat, mouse or other rodent, cat, dog, pig, sheep, goat, cattle, horse, donkey, and non-human primate). In some embodiments, the subject is a pig.

The animal may be a weaned animal. That is, an animal may be weaned prior to administration of the composition. A weaned animal has transitioned from eating solid foods, or foods instead of its mother's milk. ‘Weaning’ describes the process of transitioning an animal from its mother's milk to a diet of solid food. An animal may be in the process of weaning or may be weaned.

Different animals are typically weaned at different ages. For example, pigs are typically weaned between 2 and 8 weeks of age, while calves are typically weaned between 2 to 8 months of age. It will be appreciated that, depending on the animal, an appropriate age of animal may be chosen by the skilled person to reflect a suitably weaned animal.

The animal may be a weaned animal. The animal may be at least 2 weeks in age, preferably one of ≥3 weeks, ≥4 weeks, ≥5 weeks, ≥6 weeks, ≥7 weeks, ≥8 weeks, ≥9 weeks, ≥10 weeks, ≥11 weeks, ≥12 weeks, or ≥3 months, ≥4 months, 5 months, ≥6 months, ≥7 months, ≥8 months, ≥9 months, ≥10 months, ≥11 months, or ≥12 months, at the time they start receiving the liquid composition described herein.

The animal may be a weaned pig. The animal may be a pig of at least 14 days in age, preferably one of ≥15 days, ≥16 days, ≥17 days, ≥18 days, ≥19 days, ≥20 days, ≥21 days, or ≥3 weeks, ≥4 weeks, ≥5 weeks, ≥6 weeks, ≥7 weeks, ≥8 weeks, ≥9 weeks, ≥10 weeks, ≥11 weeks, ≥12 weeks, ≥13 weeks, ≥14 weeks, ≥15 weeks, or ≥16 weeks, at the time they start receiving the liquid composition described herein.

The animal may be a weaned calf. The animal may be a calf of at least 8 weeks in age, preferably one of ≥9 weeks, ≥10 weeks, ≥11 weeks, ≥12 weeks, or ≥3 months, ≥4 months, ≥5 months, ≥6 months, ≥7 months, ≥8 months, ≥9 months, ≥10 months, ≥11 months, or ≥12 months, at the time they start receiving the liquid composition described herein.

A subject according to the present invention may be receiving a balanced diet. That is, a subject according to the present invention may be receiving a diet that provides all essential nutrients in appropriate proportions to meet a subject's growth and maintenance requirements (including carbohydrates, fats, proteins, vitamins, minerals, fiber and water).

A subject according to the present invention may be receiving a high-fat diet, for example to promote one or more production traits. High-fat diets may contain a concentrated source of energy, such as animal fats (e.g. tallow and lard), vegetable fats (e.g. soybean oil, corn oil, canola oil), fish oil, milk fats, or protected fats.

A subject according to the present invention may be receiving a high-protein diet, for example to support health, milk production or muscle growth. High-protein diets may contain, for example, oilseed meals (e.g. soybean-meal, canola meal), high protein forages (e.g. alfalfa and clover), fish meal, distillers dried grains, pulses, cottonseed meal, or animal by-products.

A subject according to the present invention may be receiving a diet supplemented by antibiotics, for example for growth promotion, or for disease prevention. Such antibiotics may include tetracyclines, ionophores, macrolides, penicillins, aminoglycosides, bacitracin and lincosamides.

A subject according to the present invention may be receiving a diet containing additional/alternative supplements, for example to promote growth, support gut health and/or protect against infection. Such supplements may include probiotics (e.g. lactobacillus, bifidobacterium, bacillus and enterococcus species) and prebiotics (e.g. mannan-oligosaccharides, fructo-oligosaccharides, galacto-oligosaccharides and beta glucans), organic acids (e.g. formic acid, lactic acid, citric acid, butyric acid and propionic acid), phytogenics (e.g. essential oils (such as thymol, carvacrol and eugenol), herbs and spices (such as garlic, cinnamon, ginger and turmeric), saponins and tannins), enzymes (e.g. phytase, proteases, amylase, cellulase and hemicellulose, and xylanase) and amino acids (e.g. synthetic amino acids including lysine, methionine and threonine).

In some embodiments, the subject is a healthy subject, e.g. one not diagnosed with or known to be suffering from any disease or condition. A healthy subject may include subjects considered not at risk of developing any disease or condition. A healthy subject may refer to a subject not diagnosed with, or known to be suffering from, any disease or condition known to be treatable/preventable with salicylic compounds (e.g. a composition as described herein).

In some embodiments, the subject is not diagnosed with or suffering from inflammation, or a condition associated with inflammation. In some embodiments, the subject is considered not at risk of developing inflammation, or a condition associated with inflammation. In some embodiments, the subject is not diagnosed with or suffering a disease associated with excess blood clotting. In some embodiments, the subject is considered not at risk of developing a disease associated with excess blood clotting. In some embodiments, the subject is not suffering from weight loss, or a condition associated with weight loss.

In alternative embodiments, a subject to be treated may be a subject diagnosed with and/or suffering from inflammation, or a condition associated with inflammation, or is a subject considered at risk of developing such a condition. A subject may be a subject diagnosed with and/or suffering from pathological weight loss, or is a subject considered at risk of developing such a condition. That is, a subject may be a subject suffering from weight loss associated with a disease or disorder, e.g. inflammation or infection.

### Routes of Administration

The liquid composition described herein comprising the salicylate compound may be administered to a subject by any convenient route of administration, whether systemically/peripherally or topically (i.e., at the site of desired action).

Routes of administration include, but are not limited to, oral (e.g., by ingestion); buccal; sublingual; transdermal (including, e.g., by a patch, plaster, etc.); transmucosal (including, e.g., by a patch, plaster, etc.); intranasal (e.g., by nasal spray, drops or from an atomiser or dry powder delivery device); ocular (e.g., by eyedrops); pulmonary (e.g., by inhalation or insufflation therapy using, e.g., an aerosol or vapour, e.g., through the mouth or nose); rectal (e.g., by suppository or enema); vaginal (e.g., by pessary); parenteral, for example, by injection, including subcutaneous, intradermal, intramuscular, intravenous, intraarterial, intracardiac, intrathecal, intraspinal, intracapsular, subcapsular, intraorbital, intraperitoneal, intratracheal, subcuticular, intraarticular, subarachnoid, and intrasternal; by implant of a depot or reservoir, for example, subcutaneously or intramuscularly.

In one preferred embodiment, the route of administration is oral (e.g., by ingestion).

In one preferred embodiment, the route of administration is parenteral (e.g., by injection).

In one preferred embodiment, the route of administration is rectal.

In one preferred embodiment, the route of administration is nasogastric.

In one preferred embodiment, the route of administration is pulmonary (e.g. by inhalation or insufflation therapy using e.g. an aerosol or vapour through e.g. the mouth or nose).

The invention provides the use of a composition which is easily administered e.g. by the subject themselves orally or by inhalation or insufflation.

### Dosage

It will be appreciated by one of skill in the art that appropriate dosages of the compositions comprising the salicylate compound can vary from subject to subject.

For non-therapeutic applications (e.g. when used in a healthy-subject), determining the optimal dosages of the compositions will involve balancing the amount of composition required to achieve the desired effect (e.g. enhanced production trait) against any risk or deleterious side effects.

The selected dosage level will depend on a variety of factors including, but not limited to, the activity of the particular salicylate compound, the route of administration, the time of administration, the rate of excretion of the salicylate compound, the duration of the treatment, other drugs, compounds, and/or materials used in combination, the severity of the condition, and the species, sex, age, weight, condition, general health, and prior medical history of the subject. The amount of salicylate compound and route of administration will ultimately be at the discretion of the physician, veterinarian, or clinician, although generally the dosage will be selected to achieve local concentrations at the site of action which achieve the desired effect without causing substantial harmful or deleterious side-effects.

Administration can be effected in one dose, continuously or intermittently (e.g., in divided doses at appropriate intervals) throughout the course of treatment. Methods of determining the most effective means and dosage of administration are well known to those of skill in the art and will vary with the formulation used for therapy, the purpose of the therapy, the target cell(s) being treated, and the subject being treated. Single or multiple administrations can be carried out with the dose level and pattern being selected by the treating physician, veterinarian, or clinician.

In general, (e.g. for non-therapeutic applications described herein), a suitable dose of the composition described herein for the enhancement of the productivity of an animal is in the range of about 1-30 mg per kilogram body weight of the subject per day.

Where the compound is a salt, an ester, an amide, a prodrug, or the like, the amount administered is calculated on the basis of the parent compound and so the actual weight to be used is increased proportionately.

For the compositions according to the disclosure an improved bioavailability and absorption of the salicylate compound (e.g. aspirin) may be observed. Therefore, a lower dose than necessary for standard compositions may be expected to achieve the same level of therapeutic effect.

### Packaging and Kits

One aspect of the invention pertains to a kit comprising (a) a liquid composition as described herein, e.g., preferably provided in a suitable container and/or with suitable packaging; and (b) instructions for use, e.g., written instructions on how to administer the composition.

The kit may have at least one container having a predetermined quantity of the liquid composition, e.g. predetermined dose or volume.

In one embodiment, the kit further comprises one or more (e.g., 1, 2, 3, 4) additional therapeutic agents, as described herein.

The written instructions may also include a list of indications for which the active ingredient is a suitable treatment.

In some embodiments the kit may also contain apparatus suitable to administer one or more doses of the liquid composition, such apparatus preferably being provided in sterile form.

In some embodiments, the kit may include packaging manufactured by the Blow-Fill-Seal (BFS) method.

In some embodiments a method of manufacturing a kit according to the present invention may include the step of preventing water infiltration into the composition.

The liquid composition may be packaged into bottles for dispensing with a spoon or a pipette. Alternatively the composition may be packaged into a syringe, vial, or in a sachet or ‘stick shot’, such as a laminate ‘stick shot’. The composition is suitably packaged in individual 5 to 10 ml, preferably 5 ml servings.

The syringe may be a pre-filled syringe.

The liquid composition may also be incorporated in liquid gel capsules, e.g. in a dose of 37.5 mg per capsule or in a dose of one of 10-50 mg per capsule, 20-40 mg per capsule, or 30-40 mg per capsule.

Methods according to the present invention may be performed, or products may be present, in vitro, ex vivo, or in vivo. The term “in vitro” is intended to encompass experiments with materials, biological substances, cells and/or tissues in laboratory conditions or in culture whereas the term “in vivo” is intended to encompass experiments and procedures with intact multi-cellular organisms. “Ex vivo” refers to something present or taking place outside an organism, e.g. outside the human or animal body, which may be on tissue (e.g. whole organs) or cells taken from the organism.

All of the features described herein may be combined with any of the above aspects of the invention, in any combination. In addition, any upper or lower quantity or range limit used herein may be independently combined.

The invention includes the combination of the aspects and preferred features described except where such a combination is clearly impermissible or expressly avoided.

The section headings used herein are for organizational purposes only and are not to be construed as limiting the subject matter described.

Aspects and embodiments of the present invention will now be illustrated, by way of example, with reference to the accompanying figures. Further aspects and embodiments will be apparent to those skilled in the art. All documents mentioned in this text are incorporated herein by reference.

Throughout this specification, including the claims which follow, unless the context requires otherwise, the word “comprise,” and variations such as “comprises” and “comprising,” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment.

### BRIEF DESCRIPTION OF THE FIGURES

Embodiments and experiments illustrating the principles of the invention will now be discussed with reference to the accompanying figures in which:

FIG. 1 is a plot of the normalized % of salicylic acid present in a test composition against the number of days elapsed since preparation of the composition, for a composition of 90 wt % glycerol+20EO and 10 wt % aspirin. The test was conducted at 25° C.

FIG. 2 is a plot of the normalized % of salicylic acid present in a test composition against the number of days elapsed since preparation of the composition, for a composition of 89 wt % glycerol+20EO, 10 wt % aspirin and 1 wt % saccharin. The test was conducted at 25° C.

FIG. 3 is a plot of the normalized % of salicylic acid present in a test composition against the number of days elapsed since preparation of the composition, for a composition of 96.5 wt % glycerin triacetate, 2.5 wt % aspirin and 1 wt % saccharin. The test was conducted at 25° C.

FIGS. 4A to 4E. Charts showing percentage cell death with varying concentration of drug (liquid aspirin (ASP); triacetin alone (ASP vehicle); temozolomide alone (TMZ)) in ex vivo patient glioblastoma cell lines (A) SEBTA 023, (B) SEBTA 003, (C) UP 029, (D) KNS42, (E) SNF188. Studies were conducted at 96 hours post-drug treatment and show the average (+/−StDev) of three experiments conducted in triplicate. Due to solubility issues, aspirin alone was not included in this study due to an inability to treat at comparable concentrations.

FIG. 5. Representative microscopy images of treated ex vivo biopsy-derived cell lines. Images show non-treated (NT), liquid aspirin (“aspirin”) and triacetin only treatment. Contrast differences are the result of cell media pH changes following drug treatment and not due to cell confluence variation.

FIGS. 6A to 6C. (A) Chart showing percentage cell death in ex vivo biopsy-derived UP029 cells with varying concentration of drug (saccharin-only (Sac), liquid aspirin (Lqd Aspirin), triacetin-only, temozolomide-only (TMZ)). (B) and (C) Charts showing synergy analysis of ex vivo biopsy-derived UP029 cells following treatment with liquid aspirin, saccharin-only and triacetin-only. Synergy (of the liquid aspirin compound) was noted (in order to kill 30%, 50% and 70%) compared to each agent alone with the exception of triacetin-alone (at 90% reduced cell viability) at 96 hours post-treatment, where the highest dosages of triacetin alone was reducing overall cell viability.

FIGS. 7(A) to 7(F). Charts showing percentage cell death (MTS viability analysis) of control and biopsy-derived cell lines 96 hours post-treatment with varying concentration of drug (saccharin-only (Sac), liquid aspirin-only (Lqd Aspirin), triacetin-only, or temozolomide-only (TMZ)). (A) Non-neoplastic astrocytes (CC2565) included as a control, (B) SEBTA 003 (C) SEBTA 023 (D) KNS42 (E) SNF188 (F) SEBTA 025. Each plot is representative of 3 studies conducted in triplicate.

FIG. 8. Change in body weight between day 0 and day 5 in pigs either gavaged for 5 days with saline (CON) or enhanced liquid aspirin (ELA). n=8 pigs/treatment. *P<0.05 and #P<0.10 FIG. 9. Markers of intestinal integrity in pigs either gavaged for 5 days with saline (CON) or enhanced liquid aspirin (ELA). (Left panel) Ileum transepithelial electrical resistance, (Right panel) Ileum FITC-dextran transport, n=8 pigs/treatment. *P<0.05 and #P<0.10.

FIG. 10. Tight junction protein immunofluorescence images and relative fluorescence calculations of Claudin 3 (CLDN3) and Claudin 7 (CLDN7) in pigs either gavaged for 5 days with saline (CON) or enhanced liquid aspirin (ELA).

In this specification the following test method was used:

### Aspirin Stability

Aspirin undergoes hydrolysis to salicylic acid and acetic acid. The aspirin and salicylic acid concentrations in the sample composition were determined for a minimum period of at least 200 days, preferably up to a maximum of 300 days. The composition was stored in a sealed glass vial in an oven at 25° C., and the concentration of aspirin and salicylic acid measured weekly. The glass vial was opened, a sample removed for testing every week and the glass vial resealed after purging with nitrogen. High performance liquid chromatography with UV detection was used. The conditions were as follows: D Mobile phase: 40% of 1% acetic acid in water, 60% methanol.
Column: Agilent Zorbax Eclipse XBD-C18. 4.6 mm×150 mm with 5 micron particle size.
Column heater: 25° C.
Sample concentration: 0.02 g made to 10 ml with mobile phase.
Injection volume: 40 microlitre.
Flow rate: 1 ml minute.
Detection: UV at 280 nm.

The stability of the aspirin in the composition is defined as the aspirin degradation rate which was calculated as (i) the average % aspirin degradation (based on the original aspirin concentration) per day, and (ii) the average % aspirin degradation (based on the original aspirin concentration) per gram of composition per day.

The invention is illustrated by the following non-limiting examples.

### EXAMPLES

### Example 1

Food grade glycerin triacetate (ex Sigma Aldrich) was mixed with ethanol 40% w/v and passed through a fixed bed of activated earth. The solvent was then removed using vacuum distillation followed by steam distillation to levels below 1 ppm of ethanol in the glycerin triacetate.

### Example 2

A composition was prepared by mixing 2.5 wt % aspirin (ex Sigma Aldrich), 96.5 wt % glycerin triacetate (produced in Example 1), and 1 wt % saccharin (ex Sigma Aldrich). The components were mixed in the appropriate ratios and sonicated to achieve complete solution. Microscopy showed that the solution was free of any particulate material. The aspirin stability in the composition was measured weekly as described above. The aspirin degradation after 277 days was 6.9% of the original amount present, which is equivalent to a degradation rate of 0.025%/day.

### Example 3

The procedure of Example 2 was repeated except that the composition additionally contained 0.15 wt % of spearmint oil (ex Quinessence) (and correspondingly 0.15 wt % less of glycerin triacetate, i.e. 96.35 wt %). The aspirin degradation after 246 days was 5.7% of the original amount present, which is equivalent to a degradation rate of 0.023%/day.

### Example 4

A composition was prepared by mixing 2.5 wt % aspirin (ex Sigma Aldrich), 96.5 wt % glycerin triacetate (produced in Example 1), and 1 wt % saccharin (ex Sigma Aldrich). The components were mixed in the appropriate ratios and sonicated to achieve complete solution. Microscopy showed that the solution was free of any particulate material. The aspirin stability in the composition was measured weekly as described above.

The “normalised % salicylic acid” was calculated by finding the % salicylic acid in the composition, as a percentage of the total % salicylic acid and aspirin in the composition, according to the formula

Normalised % salicylic acid=(% salicylic acid)/(% aspirin+% salicylic acid)

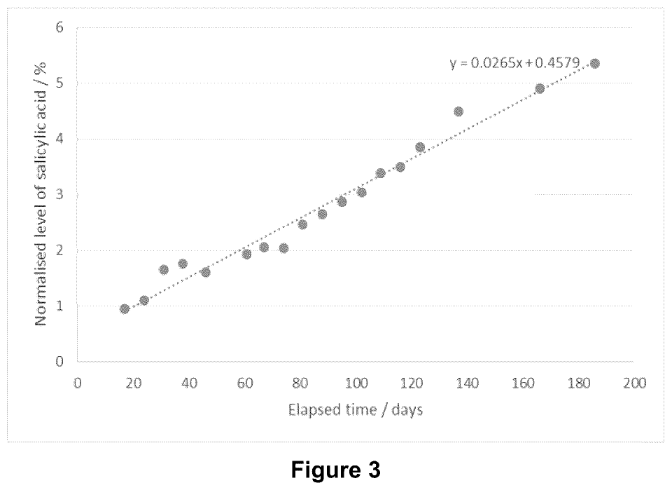

The results are shown in Table 1 below, and in FIG. 3.

TABLE 1

| Day | Normalised % salicylic acid |
|---|---|
| 17 | 0.941483 |
| 24 | 1.108112 |
| 31 | 1.644392 |
| 38 | 1.763869 |
| 46 | 1.610604 |
| 61 | 1.932933 |
| 67 | 2.056293 |
| 74 | 2.043556 |
| 81 | 2.468435 |
| 88 | 2.647186 |
| 95 | 2.868206 |
| 102 | 3.043231 |
| 109 | 3.394404 |
| 116 | 3.499079 |
| 123 | 3.856439 |
| 137 | 4.496758 |
| 166 | 4.909486 |
| 186 | 5.350264 |

The observed 5.35% degradation after 186 days is equivalent to a degradation rate of 0.0288%/day.

Extrapolating from these results, the predicted shelf life limit (the point in time after initial preparation at which the normalised % salicylic acid reaches 10%) is 360 days.

### Example 5

Compositions of aspirin with glycerin triacetate and saccharin were prepared according to the method of Example 2. Five compositions were prepared, with aspirin concentrations of 2.5 wt %, 3.0 wt %, 3.5 wt %, 4.0 wt % and 4.5 wt % respectively. Each composition also contained 1 wt % saccharin and the balance glycerin triacetate.

The compositions were tested for solubility at different temperatures. Compositions were observed to determine whether the aspirin had fully dissolved to provide a clear solution and whether the solution remained clear.

The results are shown in Table 2 below:

TABLE 2

| Aspirin | | | | | |
|---|---|---|---|---|---|
| concentration | Stable solubility at temperature | | | | |
| (wt %) | 25° C. | 20° C. | 15° C. | 10° C. | 4° C. |
| 2.5 | YES | YES | YES | YES | YES |
| 3.0 | YES | YES | YES | YES | NO |
| 3.5 | YES | YES | YES | YES | NO |
| 4.0 | YES | NO | NO | NO | NO |

The results demonstrate stable solubilisation of aspirin in the composition of the present invention at 4 wt % concentration at 25° C. At a concentration of 2.5 wt % the aspirin was stably solubilised at temperatures down to 4° C.

The compositions of the invention are therefore particularly suitable for use in the treatment of cardiovascular and cerebrovascular disorders and cancer, where low doses of aspirin over extended periods are required.

For example, at 2.5 wt % aspirin, a 2 mL dose in a gel capsule would provide around 50 mg aspirin. A 1 mL dose would provide around 25 mg aspirin.

### Comparative Example 1

A composition was prepared by mixing 10 wt % aspirin (ex Sigma Aldrich) and 90 wt % glycerol+20EO (glycerol ethoxylated with 20 mol equivalents of ethylene oxide). The components were mixed in the appropriate ratios and sonicated to achieve complete solution. Microscopy showed that the solution was free of any particulate material. The aspirin stability in the composition was measured weekly as described above.

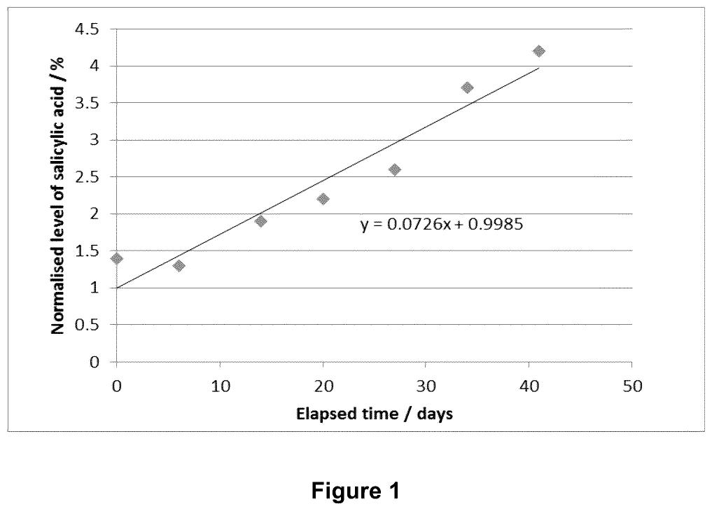

The results are shown in Table 3 below, and in FIG. 1.

TABLE 3

| Day | Normalised % salicylic acid |
|---|---|
| 0 | 1.4 |
| 6 | 1.3 |
| 14 | 1.9 |
| 20 | 2.2 |
| 27 | 2.6 |
| 34 | 3.7 |
| 41 | 4.2 |

The average degradation rate is 0.102%/day. Extrapolation from these results gives a predicted shelf life of 124 days.

### Comparative Example 2

A composition was prepared by mixing 10 wt % aspirin (ex Sigma Aldrich), 1% saccharin (ex Sigma Aldrich) and 89 wt % glycerol+20EO. The components were mixed in the appropriate ratios and sonicated to achieve complete solution. Microscopy showed that the solution was free of any particulate material. The aspirin stability in the composition was measured weekly as described above.

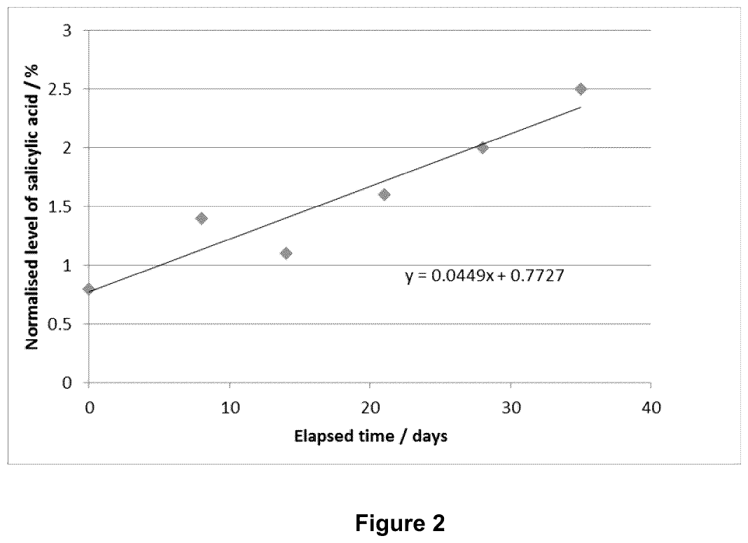

The results are shown in Table 4 below, and in FIG. 2.

TABLE 4

| Day | Normalised % salicylic acid |
|---|---|
| 0 | 0.8 |
| 8 | 1.4 |
| 14 | 1.1 |
| 21 | 1.6 |
| 28 | 2.0 |
| 35 | 2.5 |

The average degradation rate is 0.0714%/day. Extrapolation from these results gives a predicted shelf life of 205 days.

The results of the above Examples and Comparative Examples are summarised in Table 5 below:

TABLE 5

| | | Glycerin | Glycerol + | | Spearmint | Degradation | Predicted |
|---|---|---|---|---|---|---|---|
| | Aspirin | Triacetate | 20EO | Saccharin | Oil | rate | shelf-life |
| | (wt %) | (wt %) | (wt %) | (wt %) | (wt %) | (wt %/day) | (days) |
| Ex. 2 | 2.5 | 96.5 | — | 1.0 | — | 0.025 | 400 |
| Ex. 3 | 2.5 | 96.35 | — | 1.0 | 0.15 | 0.023 | 435 |
| Ex. 4 | 2.5 | 96.5 | — | 1.0 | — | 0.0288 | 360 |
| Comp. | 10 | — | 90 | — | — | 0.102 | 124 |
| Ex. 1 | | | | | | | |
| Comp. | 10 | — | 89 | 1.0 | — | 0.0714 | 205 |
| Ex. 2 | | | | | | | |

It can be seen that the degradation rate of aspirin and thereby the shelf-life of the liquid composition is improved dramatically for compositions according to the invention. Shelf life of a year or longer is possible. A comparison of Comparative Examples 1 and 2 shows that the presence of saccharin provides a significant increase in stability. Furthermore, a comparison of Comparative Example 2 with Examples 2-4 reveals a dramatic improvement in stability when the composition includes glycerin triacetate.

The above examples illustrate the improved properties of a composition according to the present invention.

### Example 6—an In Vitro Study to Investigate the Anti-Tumour Effects of Liquid Aspirin in Adult Glioblastoma, Paediatric High Grade Glioma and Medulloblastoma

### Introduction

Soluble aspirins currently on the market are in fact dispersible and therefore still contain grains that sit on the gastric mucosa causing gastric side effects. The categorization as “soluble” is therefore not accurate. Alternative aspirin products are powders that quickly disperse in water. No truly shelf stable liquid formulation of acetylsalicylic acid (ASA) has been successfully produced, until now. The unique liquid ASA described herein (referred to in this Example as ‘liquid aspirin’) is expected to show a significant reduction in gastrointestinal side effects.

As described herein, liquid aspirin contains ASA and two excipients: glycerin triacetate (triacetin) and saccharin (Sac). All three ingredients are pharmaceutically approved and have been shown to have compelling anti-tumour properties.

Triacetin has been shown to significantly augment drug delivery across the blood brain barrier (BBB), suggesting that this combination could be highly effective against glioblastoma (GBM) [Van Tellingen et al., Overcoming the blood-brain tumor barrier for effective glioblastoma treatment. Drug Resist. Updat. 19, 1-12 (2015)].

Saccharin based compounds have been proposed as a new class of anti-cancer agent (Mahon et al., Saccharin: a lead compound for structure-based drug design of carbonic anhydrase IX inhibitors. Bioorg Med Chem 2015 Feb. 15; 23(4):849-54, incorporated herein by reference). Proescholdt et al. (‘Function of carbonic anhydrase IX in glioblastoma multiforme’, Neuro Oncol, 2012, Vol. 14, pp. 1357-1366) suggest that inhibition of carbonic anhydrase IX is a potential metabolic target for the treatment of glioblastoma patients.

Of the three components, aspirin has demonstrated the most potent anti-tumour effect, particularly against GBM. An initial in vivo study highlighted that the administration of aspirin into an established Fischer 344 rat glioma model (Aas, A. T., Brun, A., Blennow, C., Stromblad, S., & Salford, L. G. The RG2 rat glioma model. J. Neurooncol. 23, 175-183 (1995)) significantly inhibited the growth of differentiated malignant glioblastoma RG2 cells both when administered the day before tumour cell inoculation as well as in established rat glioblastoma tumours (Aas, A. T., Tonnessen, T. I., Brun, A., & Salford, L. G. Growth inhibition of rat glioma cells in vitro and in vivo by aspirin. J. Neurooncol. 24, 171-180 (1995)).

Prostaglandin E2 (PGE2) has been shown to have an important role in both immunosuppression and tumour growth. As a PGE2 inhibitor, aspirin has been shown to reduce in vitro tumour cell proliferation. Aspirin dosage studies were conducted, evaluating the effect of both high and low dose aspirin exposure on PGE2 synthesis in the in vitro C₆ glioma model. These studies revealed that aspirin directly inhibited PGE2 synthesis in C6 cells and that critically, low-dose aspirin is as effective as high-dose aspirin in mediating this response (Hwang, S. L. et al. Effect of aspirin and indomethacin on prostaglandin E2 synthesis in C6 glioma cells. Kaohsiung. J. Med. Sci. 20, 1-5 (2004)).

Human A172 glioblastoma cells treated with aspirin induced significant apoptosis (programmed cell death) [Kim, S. R. et al. Aspirin induces apoptosis through the blockade of IL-6-STAT3 signaling pathway in human glioblastoma A172 cells. Biochem. Biophys. Res. Commun. 387, 342-347 (2009)]. The underlying mechanism for this response was a reduction in the level of phosphorylated signal transducer and activator of transcription 3 (STAT3), specifically pTyr-STAT3. STAT3 is a transcription factor that is required for survival of A172 cells. This conclusion was supported by measuring cyclin D1, XIAP, and Bcl-2 transcription that was notably attenuated after aspirin treatment (Kim et al., supra). Implicating STAT3 further, the expression and secretion of interleukin-6 (IL-6) (that induces STAT3 phosphorylation), was notably inhibited by aspirin treatment (Kim et al., supra).

Drawing from these findings, it is known that hypoxia can activate STAT3 and subsequently induce angiogenesis (the development of blood vessels) [Greten, F. R. & Karin, M. Peering into the aftermath: JAKi rips STAT3 in cancer. Nat. Med. 16, 1085-1087 (2010)]. In most solid malignancies, persistent STAT3 signalling is triggered by an autocrine-paracrine production of IL-6 that is noticeably higher in a hypoxic environment (Song, Y. Y. et al. STAT3, p-STAT3 and HIF-1alpha are associated with vasculogenic mimicry and impact on survival in gastric adenocarcinoma. Oncol. Lett. 8, 431-437 (2014)). Hypoxia is a hallmark of GBM, with tumours showing pseudopalisades of neoplastic cells surrounding areas of frank necrosis as well as signs of vascular proliferation. These areas of peri-necrotic hyper-cellularity have been well characterized and are not the result of increased proliferation. As one would predict, these regions have high levels of hypoxia-induced factor 1 alpha (HIF1α) expression, resulting in pro-angiogenic vascular endothelial growth factor (VEGF) secretion as well as elevated IL-6. This in turn drives vascular proliferation. However, the vessels that are generated in response to VEGF within this environment are severely malformed (Jain, R. K. Normalizing tumor microenvironment to treat cancer: bench to bedside to biomarkers. J. Clin. Oncol. 31, 2205-2218 (2013)). The result is deregulated vessel structure with gaps between endothelial cells and an absence of pericytes. Due to this malformation and inherent leakiness, the interstitial pressure is increased resulting in vascular stasis with corresponding exacerbation of hypoxia and increased microvascular thrombosis (Jain et al., supra). Strikingly, it has been shown that aspirin selectively suppresses inflammation, and specifically IL-6-induced T-helper cell 17. This mediates the down regulation of acetyl-STAT3 expression as well as blocking IL-17A-induced inflammation and IL-6 production. This reduction of IL-6 production will then result in a concomitant reduction in active (phosphorylated) STAT3.

More recently, it has been shown that aspirin represses the transcriptional activity of the β-catenin/TCF protein complex. As a consequence of this, aspirin directly inhibits GBM proliferation and invasion as well as inducing apoptosis (Jin, T., George, F., I, & Sun, J. Wnt and beyond Wnt: multiple mechanisms control the transcriptional property of beta-catenin. Cell Signal. 20, 1697-1704 (2008)). The results presented within this study suggest that aspirin exerts its anti-neoplastic action by suppressing the β-catenin/TCF signalling pathway in GBM. This is particularly striking as recent data has highlighted that FoxM1 promotes the development and progression of GBM by regulating key factors involved in cell division, epithelial to mesenchymal transition (EMT), invasion, angiogenesis and upregulation of the Wnt/β-catenin signalling network (Wang, Z., Zhang, S., Siu, T. L., & Huang, S. Glioblastoma multiforme formation and EMT: role of FoxM1 transcription factor. Curr. Pharm. Des 21, 1268-1271 (2015)). A deregulated Wnt/β-catenin network has been reported in GBM and it has been suggested that this could also constitute a therapeutic target (Zhang, K. et al. ICAT inhibits glioblastoma cell proliferation by suppressing Wnt/beta-catenin activity. Cancer Lett. 357, 404-411 (2015)).

### Experimental Method and Results

Many of the established glioma cell lines, such as C6, A172, U87, U373 and U251 have been grown/passaged in research laboratories around the world for many years and, as a consequence, display a high degree of cellular heterogeneity rendering them increasingly dissimilar to their original primary/early passage cultures and, indeed, from the biopsy from which they were derived.

To address this issue, we used a panel of patient-derived (adult and paediatric) ex vivo GBM low passage cell cultures, which have been characterized at the molecular level, including DNA fingerprinting (Prof. G. Pilkington, Brain Tumour Research Centre, University of Portsmouth, UK). These cells are truly representative of the patient GBM and, as a result, any anti-tumour effect observed will have clinical relevance. This extensive cell culture bio-bank allows screening of novel anti-GBM therapeutics prior to in vivo studies and clinical trials.

It is also critical to compare these novel therapeutics against the currently prescribed frontline chemotherapeutics. Consequently, our studies also include temozolomide, which is a standard of care treatment for GBM.

Non-neoplastic astrocytes provide a control element within these studies. These are non-cancerous and any proposed therapeutic will preferably have selective anti-cancer action (i.e. not kill normal, non-tumor, cells).

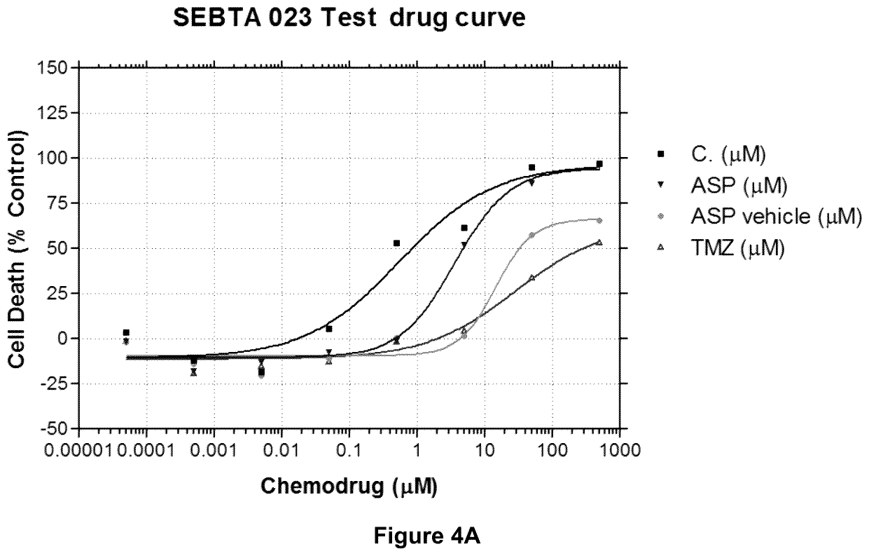

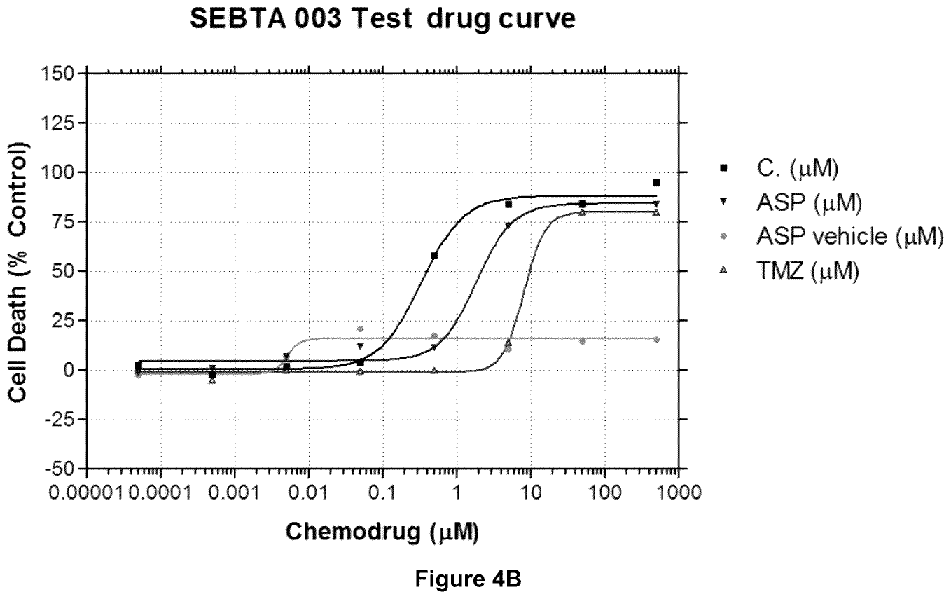

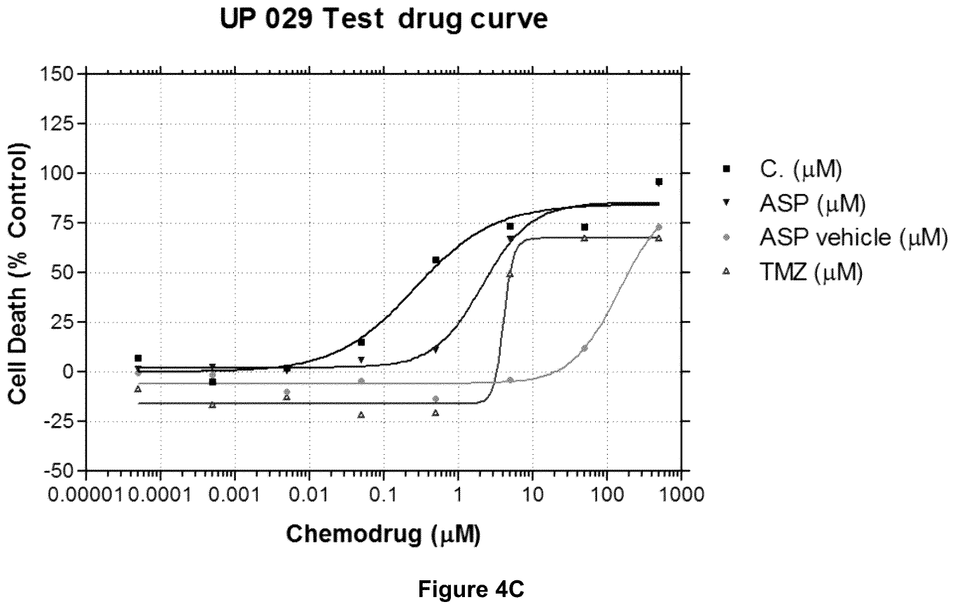

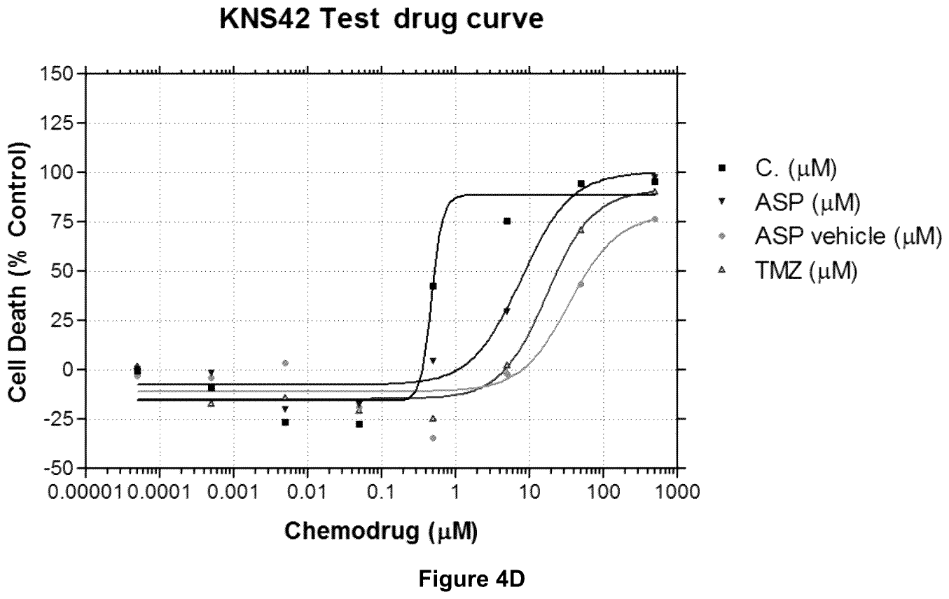

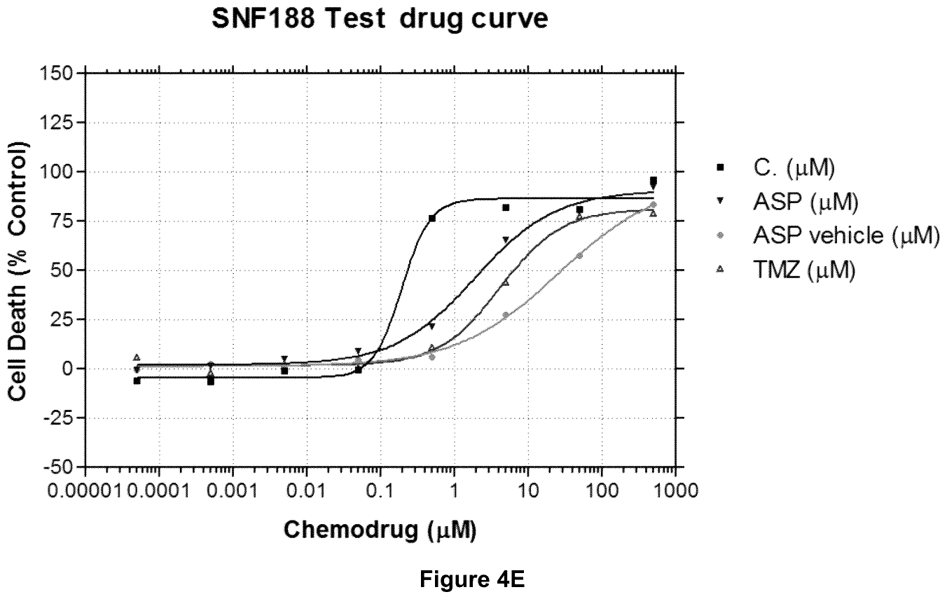

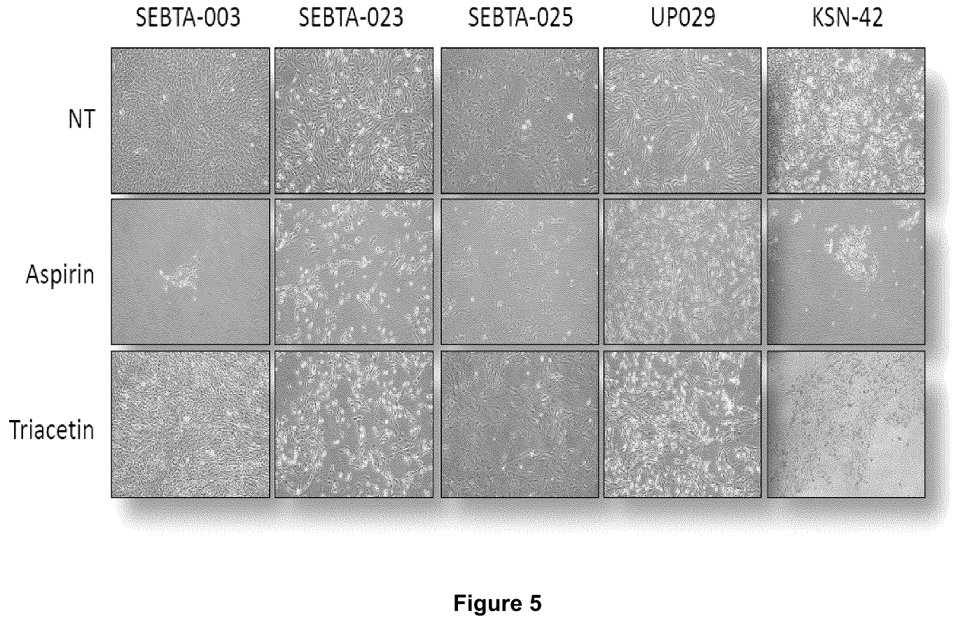

Viability studies were conducted to compare the effect of liquid aspirin (ASP), triacetin (ASP vehicle), and temozolomide (TMZ) in five adult GBM ex vivo cell lines from our panel. Results are shown in FIGS. 4A to 4E. Liquid aspirin showed notably better induction of cell death in all GBM cells tested.

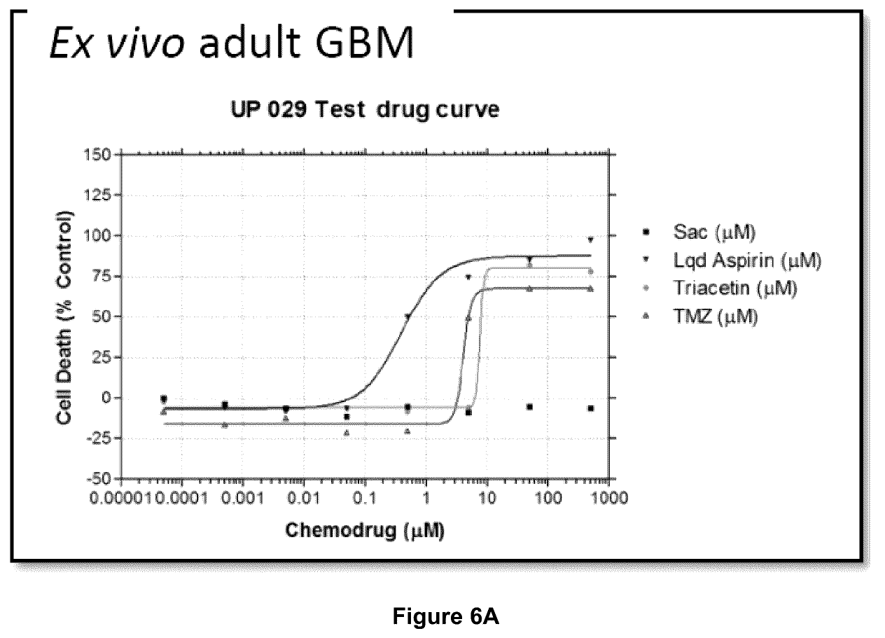

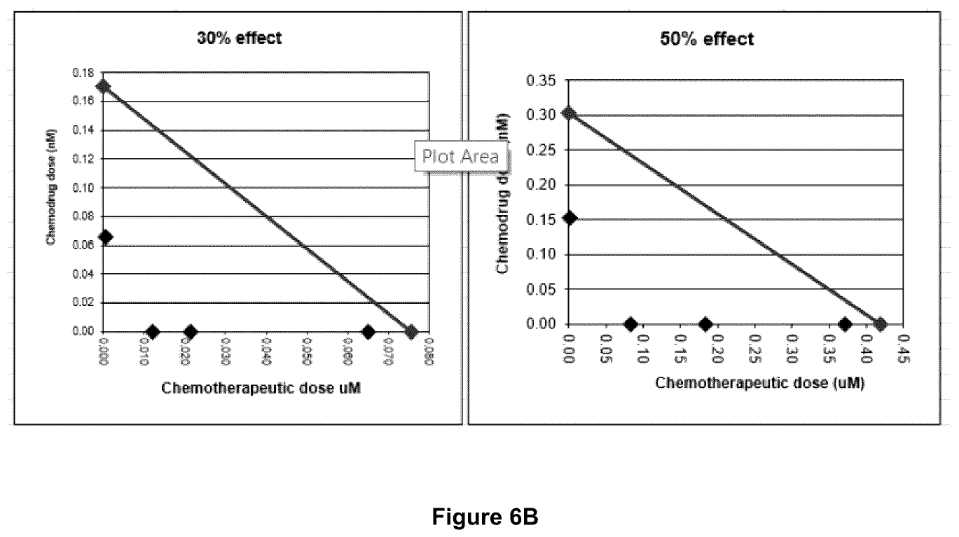

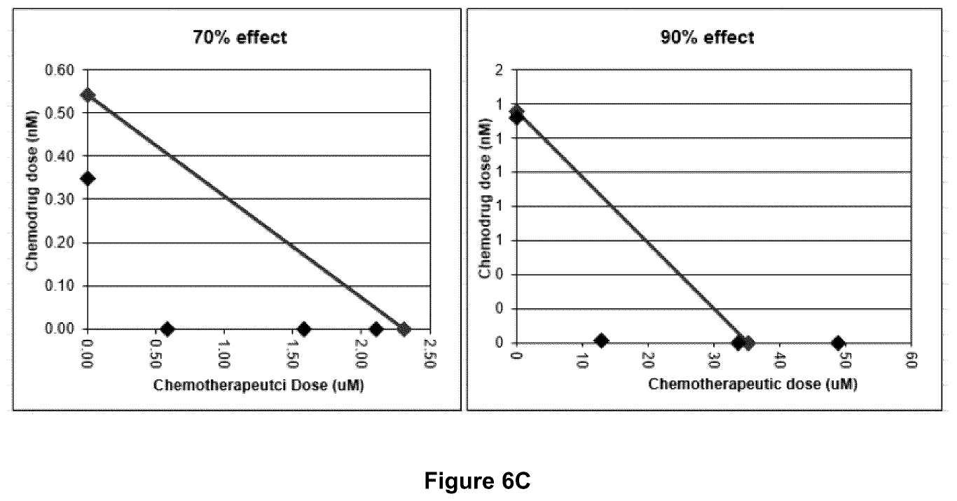

Synergy studies were also conducted to directly address the specific anti-tumour efficacy of each of aspirin, triacetin and saccharin (Sac) as individual components and the triple-formulation of aspirin, triacetin and saccharin (liquid aspirin). We have significant experience conducting this type of analysis and importantly can differentiate between additive effects versus a synergistic response (Hallden, G. et al. Novel immunocompetent murine tumor models for the assessment of replication-competent oncolytic adenovirus efficacy. Mol. Ther. 8, 412-424 (2003); Cheong, S. C. et al. E1A-expressing adenoviral E3B mutants act synergistically with chemotherapeutics in immunocompetent tumor models. Cancer Gene Ther. 15, 40-50 (2008)). FIGS. 6B and 6C show synergy of liquid aspirin in order to kill 30%, 50% and 70% of UP 029 cells compared to use of each agent alone with the exception of triacetin-alone at 90% reduced cell viability, where the highest dosages of triacetin alone reduced overall cell viability.

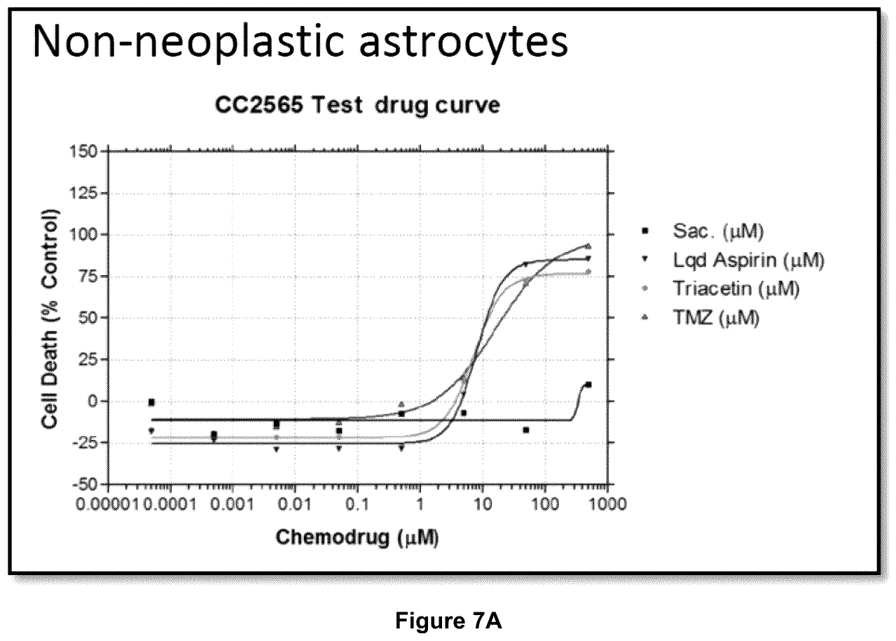

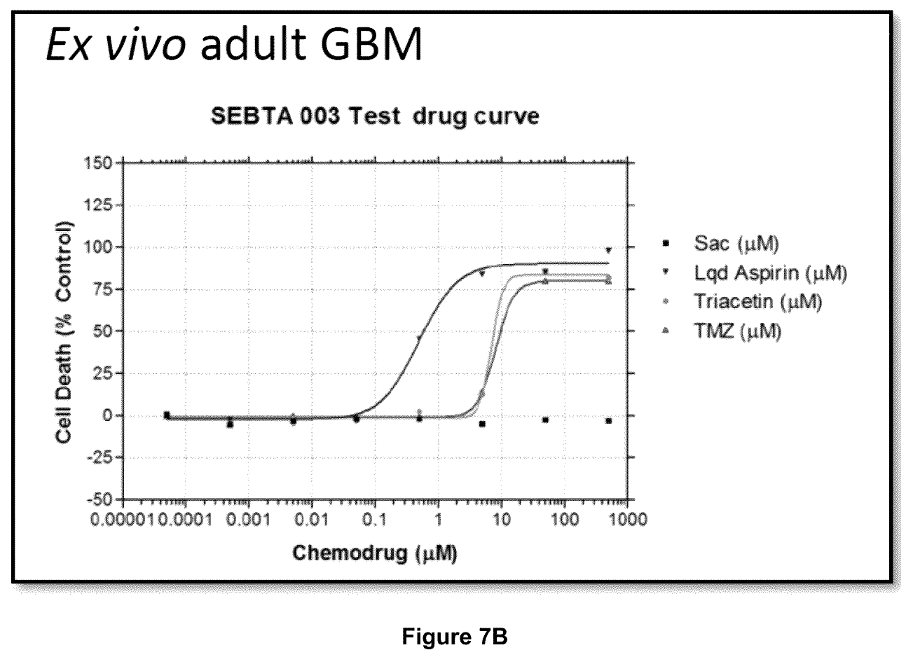

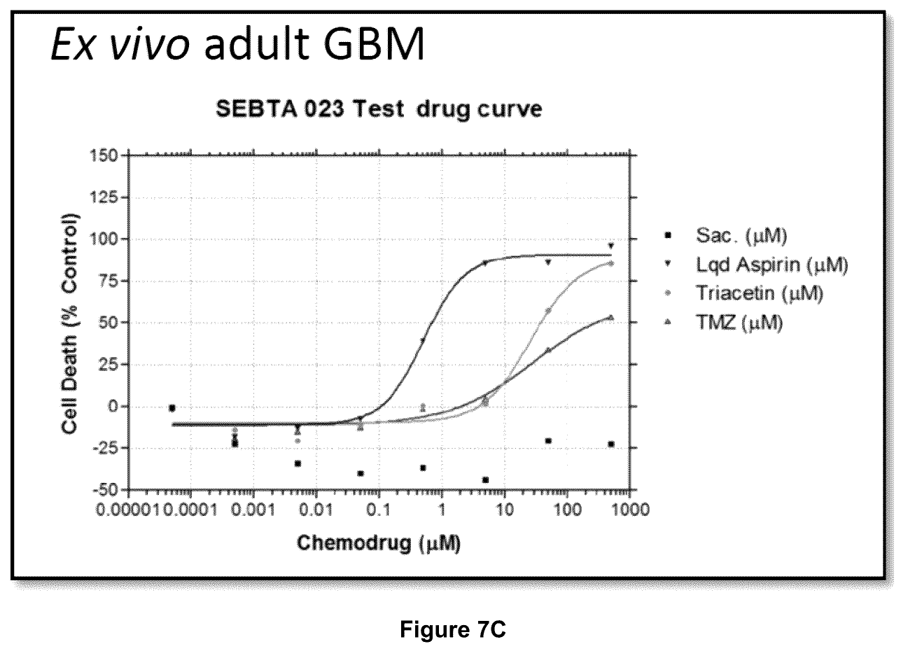

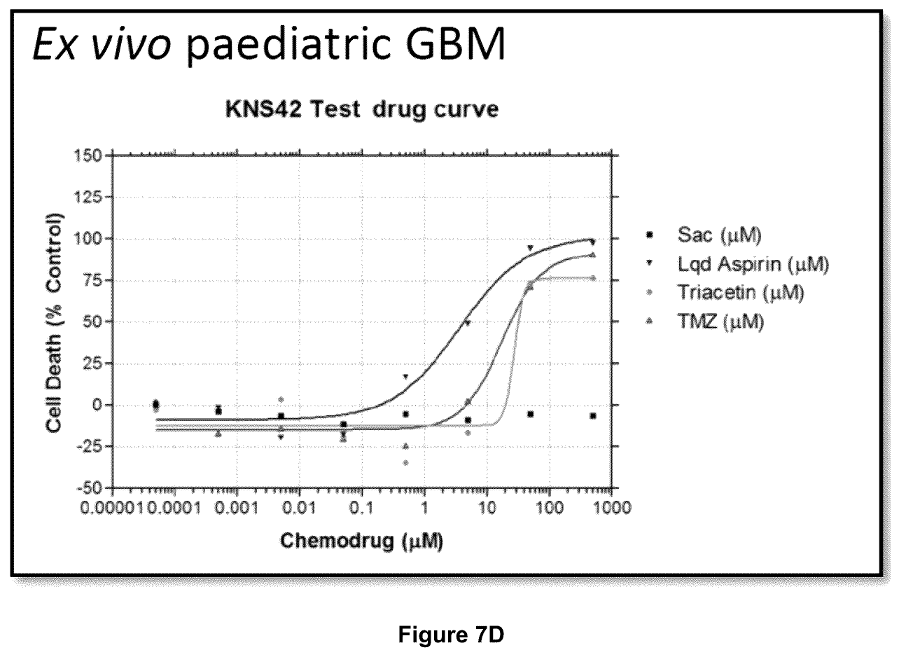

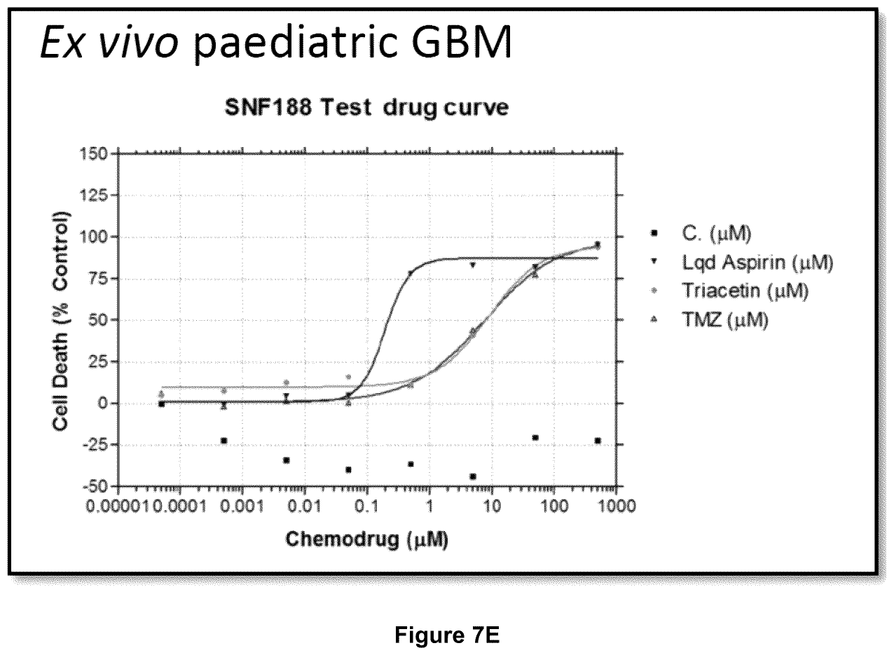

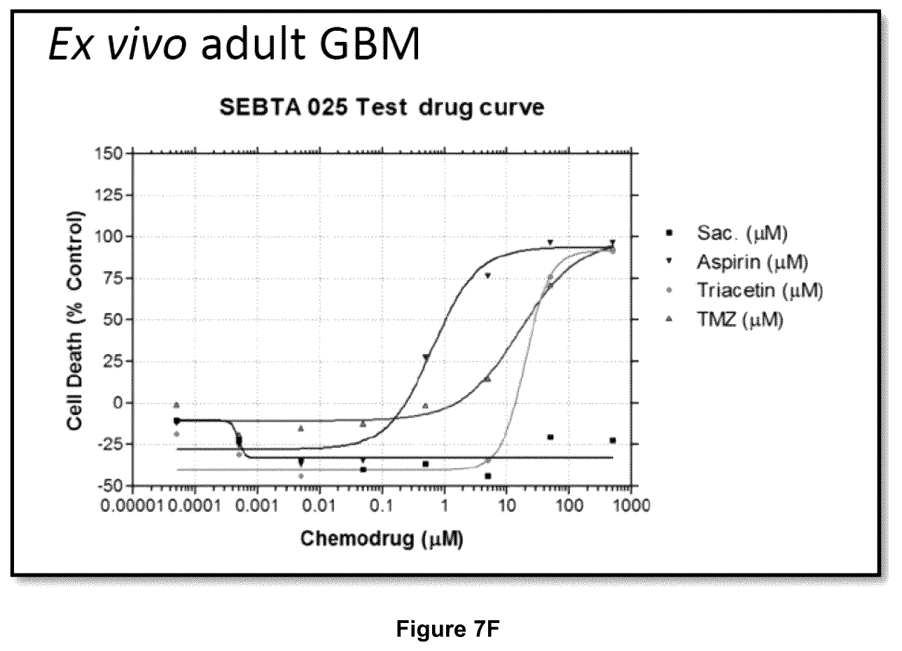

We also confirmed that liquid aspirin demonstrates anti-cancer specificity, i.e. is not toxic to non-neoplastic astrocytes. Results are shown in FIG. 7A to 7F which show liquid aspirin to have markedly better cell killing ability than triacetin or temozolomide; liquid aspirin typically having at least one order of magnitude greater potency in inducing cell death than triacetin or temozolomide. Liquid aspirin had significantly less toxicity when added to non-neoplastic cells (FIG. 7A). Indeed, the associated toxicity noted within the astrocyte cell line could be associated with triacetin-alone (which demonstrated a similar MTS-viability profile).

### Example 7—an In Vivo Study to Investigate the Effects of Liquid Aspirin on Growth in Animals

### Materials and Methods

All animal procedures were approved by the Iowa State University Institutional Animal Care and Use Committee and adhered to the guidelines for ethical and humane use of animals for research.

### Experimental Design and Diets

Sixteen barrows (n=8/treatment) were blocked by body weight (initial BW 6.2 kg) and divided into two treatment groups; control and ELA. Pigs were housed in group pens and fed the same diet (Table 6), but individually orally gavaged saline or ELA (comprising aspirin at a concentration of 25 mg/ml) at 2 mg/kg body weight every day for 5 d. Pig BW were recorded on DO and D5. At the end of the experiment (d 14 post-weaning), pigs were euthanized using captive bolt followed by exsanguination for sample collection.

TABLE 6
Phase 1 Diets

| Ingredient | % |
|---|---|
| Corn | 50.40 |
| SBM; 45.8% | 26.20 |
| HP300 | 8.00 |
| Soybean oil | 1.21 |
| Limestone | 0.82 |
| Monocal phosphate | 0.56 |
| Salt | 0.50 |
| Whey permeate; | 10.53 |
| (16% DM as fed) | |
| Lysine HCl | 0.46 |
| DL Methionine | 0.25 |
| L Threonine | 0.19 |
| L Tryptophan | 0.04 |
| L Valine | 0.03 |
| Zinc Oxide | 0.40 |
| Quantum Blue 5G | 0.04 |
| Trace Mineral Premix Piglet | 0.15 |
| Vitamin Premix Piglet | 0.23 |
| 12-20 lbs | 100 |

### Sample Collection

Following euthanasia, ileum (36 cm proximal from the cecum) was collected. Tissue was snap frozen and tissue were flushed with Krebs-Henseleit buffer (KB, 25 mM NaHCO₃, 120 mM NaCl, 1 mM MgSO₄, 6.3 mM KCl, 2 mM CaCl₂), and 0.32 mM NaH₂PO₄, pH 7.4) and stored at −80° C. until further analysis. Additional samples were also placed in formalin and then transferred to ethanol for histological analysis.

### Intestinal Integrity and Nutrient Transport (Ussing Chambers)

Fresh ileum segments were mounted into modified Ussing chambers (Physiological Instruments, San Diego, CA) to assess measures of ileal barrier integrity and active nutrient transport. Additionally, ex vivo ileal mucosal to serosal bacterial translocation was assessed. Immediately following euthanasia, freshly isolated intestinal sections from the distal ileum were flushed with KB and then placed in aerated bottles containing KB. Tissues were pinned and placed vertically into modified Ussing chambers, connected to dual channel voltage and current electrodes submerged in 3% noble agar, and filled with 3 M KCl to provide electrical conductance. The serosal side was bathed in KB containing 100 μg/mL ampicillin and the mucosal side was bathed in KB. Each ileum segment was clamped at a voltage of 0 mV and ileal transepithelial electrical resistance (TER) as previously described (Gabler et al., 2007). Ex vivo permeability was also assessed by 4 kDa FITC-dextran flux as previously described (Helm et al., 2020).

### Inflammation

Protein was extracted from one homogenous ground frozen ileum per pig using Tissue Protein Extraction Reagent (T-PER) with protease and phosphatase inhibitors (Thermo Fisher Scientific, Waltham MA). Protein concentration was then analyzed using the bicinchoninic acid assay (Thermo Fisher Scientific, Waltham MA). Cytokine concentrations in protein extracts were analyzed in duplicate using a commercially available kit (Porcine Cytokine and Chemokine MILLIPLEX, Millipore Sigma, St. Louis, MO) and were run on a multiplexing image analyzer (MAGPIX, Luminex, Austin, TX). Cytokines analyzed included Granulocyte-macrophage colony-stimulating factor (GM-CSF), Interferon-γ (IFNγ), as well as Interleukins IL-1a, IL-1β, IL-1ra, IL-2, IL-4, IL-6, IL-8, IL-10, IL-12, IL-18 and Tumor necrosis factor-α (TNFα). GM-CSF, IFNγ, and TNFα were not detectable in tissue samples thus data is not presented.

### Intestinal Morphology

Formalin fixed tissues of ileum and colon were paraffin-embedded into blocks, sectioned and stained with hematoxylin and eosin for evaluation of intestinal morphology as previously described (Helm et al., 2020). Briefly, images were taken at 20× magnification using a DP80 Olympus Camera mounted on an OLYMPUS BX 53/43 microscope (Olympic Scientific, Waltham, MA). In ileal sections, 15-20 well-orientated villus and crypt pairs were measured for height and depth, respectively, in μm using OLYMPUS CellSens Dimension 1.16 software (Olympus Scientific, Waltham, MA). Villus to crypt ratios were calculated and reported. In colon sections, 15-20 crypts per pig were measured for depth as described above.

### Immunofluorescence

Blank slides were stained with Claudin 3 (CLDN3; Thermo Fisher Scientific, Emeryville, CA) and Claudin 7 (CLDN7; Thermo Fisher Scientific, Emeryville, CA) per methods described in (Zaqout et al., 2020). Samples were also stained for nuclei with DAPI (4′,6-diamidino-2-phenylindole) at 1:1000. Slides were imaged on an Olympus BX63 automated fluorescence microscope using the multichannel and z-stack functions (Evident Scientific, Waltham, MA). Fluorescence values correlated to protein expression were analyzed using the corrected total cell fluorescence (CTCF) method where CTCF=integrated density −(area of selected marker×mean fluorescence of the background) and then calculated relative to controls (CON) using ImageJ version 1.8, NIH as previously described (Pearce et al., 2022).

### Multiplex Gene Assay

The QuantiGene™ Plex Sample Processing Kit (Thermofisher Scientific, Waltham, MA) was utilized to process whole ileum tissue samples. Briefly, 50 mg of powdered ileum tissue and 1 mL of working homogenization solution were added to a 2-mL microcentrifuge tube containing silica beads. Samples were loaded into a bead beater for 3 min at maximum speed. Homogenized samples were then incubated at 65° C. on a heat block for 30 min. Each sample was vortexed at maximum speed for 1 min every 10 min during incubation. Samples were centrifuged at 16,000×g for 15 min to pellet remaining cellular debris and supernatant was transferred to a 1.5 mL microcentrifuge tube for storage. Supernatants were then analyzed on a QuantiGene™ Plex Gene Expression Array (ThermoFisher Scientific, Waltham, MA). Samples were lysed at 37° C. for 30 min and then diluted 1:5 with homogenization solution. A working bead mix containing, in order, nuclease-free water, lysis mixture, blocking reagent, proteinase K, capture beads, and probe set was then prepared. The working bead mix was vortexed and added into a hybridization plate with diluted tissue homogenate, sealed, and placed overnight at 54° C. at 600 rpm. The following day samples were transferred from a hybridization plate to the magnetic separation plate and wells were washed 3×. Pre-amplifier solution was then added and placed at 50° C. at 600 rpm for 1 h. Next, the plate was washed 3× and amplifier solution was added and placed at 50° C. at 600 rpm for 1 h. The plate was then washed, and label probe solution was added for 1 h at 50° C. at 600 rpm and then washed again.

Proprietary probes were design for Sus scrofa by Thermosteric Scientific (Waltham, MA) and include the genes: atonal BHLH transcription factor 1 (ATOH1), BM11 proto-oncogene, polycomb ring finger (BMI1), cyclin dependent kinase inhibitor 1B (CDKN1B), cyclin dependent kinase inhibitor 1C (CDKN1C), chromogranin A (CGA), claudin-2 (CLDN2), claudin-3 (CLDN3), claudin-4 (CLDN4), catenin beta 1 (CTNNB1), defensin beta 1 (DEFB1), fatty acid binding protein 2 (FABP2), free fatty acid receptor 3 (FFAR3), ghrelin (GHRL), beta-glucuronidase (GUSB), Hes family BHLH transcription factor 1 (HES1), hypoxanthine phosphoribosyltransferase 1 (HPRT1), interferon-γ (IFNG), interleukin-1pR (IL1B), interleukin 1 receptor agonist (IL1 RN), interleukin 10 (IL10), interleukin 17A (IL17A), junctional adhesion molecule 2 (JAM2), lactase (LCT), leucine rich repeat containing G protein coupled receptor 5 (LGR5), lysozyme (LYZ), microtubule actin cross-linking factor 1 (MACF1), mucin 2 (MUC2), mucin 5AC (MUC5AC), myosin light chain kinase (MYLK), notch receptor 1 (NOTCH1), occludin (OCLN), phosphoglycerate kinase (PGK1), regenerating family member 3 gamma (REG3G), ribosomal protein L32 (RPL32), sucrase isomaltase (SI), solute carrier family 1 member 2 (SLC1A2), solute carrier family 2 member 5 aka glucose transporter 5 (SLC2A5/GLUT5),), solute carrier family 5 member 1 aka sodium/glucose transporter 1 (SLC5A1/SGLT1), solute carrier family 6 member 19 also known as sodium-dependent neutral amino acid transporter B(0)AT1 (SLC6A19/BOAT1), solute carrier family 27 member 4 aka long-chain fatty acid transporter protein 4 (SLC27A4),), solute carrier family 38 member 2 also known as sodium-coupled neutral amino acid transporter 2 (SLC38A2/SNAT2), SRY-box transcription factor 9 (SOX9), trefoil factor 2 (TFF2), trefoil factor 3 (TFF3), tight junction protein 1 (TJP1), tumor necrosis factor (TNF), toll-like receptor 2 (TLR2), toll-like receptor 3 (TLR3), toll-like receptor 4 (TLR4), and wingless-type MMTV integration site family, member 3A (WNT3A).

Finally, Streptavidin R-Phycoerythrin Conjugate (SAPE) working reagent was added and the plate was covered with foil for 30 min at room temperature. Afterwards, SAPE wash buffer was added and the plate was run on a Luminex MAGPIX Instrument (Luminex, Northbrook, IL). Data were analyzed on the QuantiGene™ Plex Analysis Software using the GEOMEAN of multiple housekeeping genes to generate normalized expression of values of target genes (ThermoFisher Scientific, Waltham, MA) where housekeeping genes included HPRT1, RPL32, and GUSB.

### Results

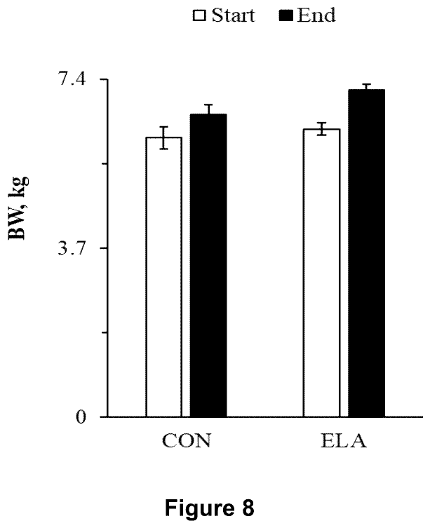

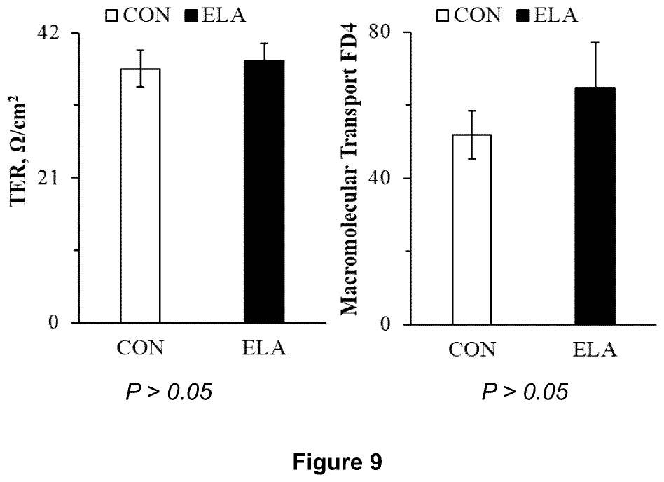

Initial BW was not different between treatments (P>0.05; FIG. 8). However, at the end of the 5-d experimental period, ELA pigs had a significantly higher BW compared to CON pigs (P<0.05; FIG. 8) where CON pigs exhibited an 8% increase in BW from D0-D5 while ELA pigs exhibited a 13.7% increase in BW from D0-D5.

Intestinal integrity as measured by TER and FITC-Dextran transport was not significantly different between treatment groups (P>0.05; FIG. 9). Intestinal inflammation in the ileum was not significantly different between treatment groups for IL-1A, IL-1B, IL1-ra, IL-2, IL-4, IL-6, IL-8, IL-10, IL-12 or IL-18 (P>0.05; Table 7).

TABLE 7
Ileum Cytokine Concentrations

| Cytokine, pg/mL | CON | ELA | P-value |
|---|---|---|---|
| IL-1A | 0.04 ± 0.005 | 0.06 ± 0.005 | >0.05 |
| IL-1B | 0.94 ± 0.168 | 0.84 ± 0.140 | >0.05 |
| IL-1ra | 0.54 ± 0.046 | 0.63 ± 0.056 | >0.05 |
| IL-2 | 0.03 ± 0.012 | 0.01 ± 0.004 | >0.05 |
| IL-4 | 0.08 ± 0.047 | 0.03 ± 0.012 | >0.05 |
| IL-6 | 0.03 ± 0.006 | 0.03 ± 0.008 | >0.05 |
| IL-8 | 14.60 ± 3.12 | 15.34 ± 3.04 | >0.05 |
| IL-10 | 0.07 ± 0.031 | 0.03 ± 0.011 | >0.05 |
| IL-12 | 0.07 ± 0.012 | 0.08 ± 0.015 | >0.05 |
| IL-18 | 23.18 ± 2.72 | 20.57 ± 2.47 | >0.05 |

n = 8 animals per treatment
Samples were run in duplicate
P < 0.05 = significant

There was tendency for an increase of 24% in villus height in ELA pigs compared to CON (P=0.06; Table 8) and a significant decrease of 25% in crypt depth in ELA-treated pigs compared to CON (P<0.05; Table 8).

TABLE 8
Ileum Morphology

| Morphology | CON | ELA | P-Value |
|---|---|---|---|
| Villus Height, μm | 247.9 ± 19.9 | 308.4 ± 21.3 | 0.06 |
| Crypt Depth, μm | 160.7 ± 9.9 | 119.4 ± 9.9 | <0.05 |
| Villus Width | 105.0 ± 3.9 | 106.4 ± 3.9 | 0.81 |

n = 8 animals/treatment
2 fields of view with a total of 10 villi measured per pig
P < 0.05 = significant
P < 0.05 = tendency

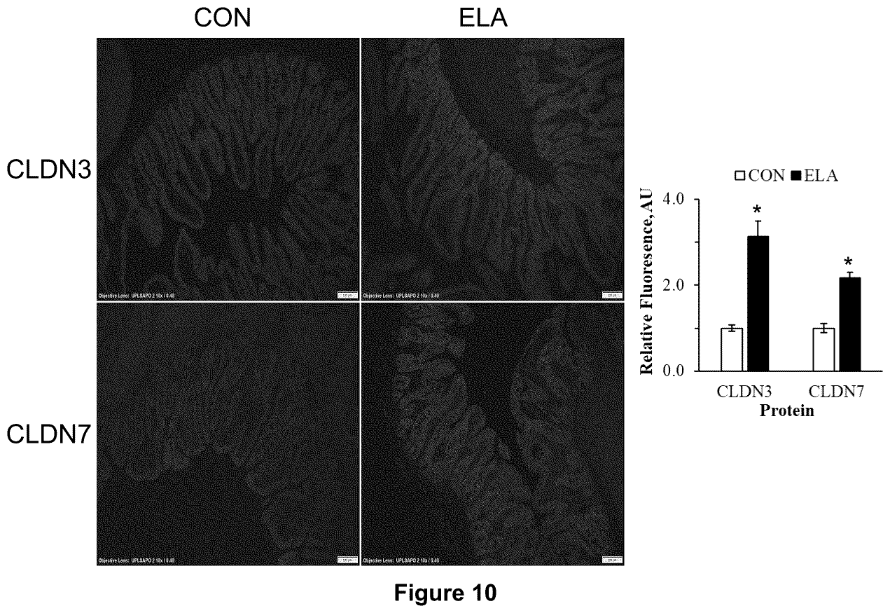

Corrected fluorescence of proteins CLDN3 were increased in pigs where CLDN3 was 3-fold higher and CLDN7 was 2-fold higher (P<0.05; FIG. 10).

Markers for AMCF-1, ATOH1, BMI1, CGA, CLDN3, CTNNB1, FABP2, FFAR3, GHRL, HES1, IL10, IL17, JAM2, LYZ, MUC2, MUCSAC, OCLN, SI, SLC27A4, SLCA2, SLC38A2, SLC5A1, SLC6A19, TFF2, TFF3, TLR3, TLR4, and TNF were not statistically different between treatment groups (P>0.05; Table 9). Tight junction gene CLDN4 was significantly increased in the ELA treated group (P=0.01). There was a tendency for immune marker IL1 RN (P=0.076) to be increased in the ELA group while TLR2 tended to decrease (P=0.080). There was tendency for stem cell markers LGR5 (P=0.086) and SOX9 (P=0.060; Table 9) to be increased in the ELA treated group. CDKN1B was significantly decreased in ELA pigs while CDKN1C was significantly increased (P≤0.05). Tight junction genes CLDN2 and MYLK were significantly increased (P≤0.05, Table 8) while TJP1 was significantly decreased (P≤0.05). DEFB1, IFNG, IL1B, LCT, REG3G, NOTCH1, WNT3A.

TABLE 4
Relative Ileum Gene Expression

| Gene | CON | ELA | SE | P-Value |
|---|---|---|---|---|
| AMCF-1 | 1.0 | 1.09 | 0.459 | 0.87 |
| ATOH1 | 1.0 | 0.76 | 0.405 | 0.68 |
| BMI1 | 1.0 | 0.99 | 0.309 | 0.98 |
| CDKN1B | 1.0 | 0.72 | 0.040 | 0.003 |
| CDKN1C | 1.0 | 1.58 | 0.147 | 0.01 |
| CGA | 1.0 | 12.89 | 10.968 | 0.34 |
| CLDN2 | 1.0 | 2.24 | 0.237 | 0.002 |
| CLDN3 | 1.0 | 2.82 | 1.356 | 0.25 |
| CLDN4 | 1.0 | 1.50 | 0.238 | 0.01 |
| CTNNB1 | 1.0 | 1.06 | 0.034 | 0.20 |
| DEFB1 | 1.0 | 0.47 | 0.138 | 0.01 |
| FABP2 | 1.0 | 1.35 | 0.270 | 0.32 |
| FFAR3 | 1.0 | 1.47 | 0.229 | 0.15 |
| GHRL | 1.0 | 6.87 | 5.528 | 0.35 |
| HES1 | 1.0 | 1.05 | 0.044 | 0.56 |
| IFNG | 1.0 | 0.09 | 0.012 | 0.001 |
| IL10 | 1.0 | 0.76 | 0.168 | 0.27 |
| IL17A | 1.0 | 1.02 | 0.119 | 0.90 |
| IL1B | 1.0 | 0.85 | 0.297 | 0.02 |
| IL1RN | 1.0 | 3.11 | 1.433 | 0.08 |
| JAM2 | 1.0 | 1.02 | 0.092 | 0.88 |
| LCT | 1.0 | 0.13 | 0.017 | 0.04 |
| LGR5 | 1.0 | 1.28 | 0.107 | 0.09 |
| LYZ | 1.0 | 1.83 | 0.455 | 0.18 |
| MUC2 | 1.0 | 0.81 | 0.101 | 0.17 |
| MUC5AC | 1.0 | 1.03 | 0.096 | 0.79 |
| MYLK | 1.0 | 2.17 | 0.265 | 0.01 |
| NOTCH1 | 1.0 | 0.84 | 0.039 | 0.05 |
| OCLN | 1.0 | 1.05 | 0.130 | 0.34 |
| REG3G | 1.0 | 3.90 | 1.135 | 0.05 |
| SI | 1.0 | 0.84 | 0.120 | 0.42 |
| SLC27A4 | 1.0 | 1.31 | 0.277 | 0.36 |
| SLC2A2 | 1.0 | 1.38 | 0.341 | 0.38 |
| SLC2A5 | 1.0 | 2.37 | 0.639 | 0.18 |
| SLC38A2 | 1.0 | 0.83 | 0.101 | 0.24 |
| SLC5A1 | 1.0 | 0.86 | 0.138 | 0.39 |
| SLC6A19 | 1.0 | 1.46 | 0.240 | 0.12 |
| SOX9 | 1.0 | 1.18 | 0.068 | 0.06 |
| TFF2 | 1.0 | 1.60 | 0.404 | 0.26 |
| TFF3 | 1.0 | 0.88 | 0.139 | 0.54 |
| TJP1 | 1.0 | 0.77 | 0.060 | 0.05 |
| TLR2 | 1.0 | 0.49 | 0.125 | 0.08 |
| TLR3 | 1.0 | 1.01 | 0.089 | 0.48 |
| TLR4 | 1.0 | 2.09 | 0.955 | 0.32 |
| TNF | 1.0 | 0.78 | 0.184 | 0.33 |
| WNT3A | 1.0 | 0.66 | 0.082 | 0.028 |

n = 8 animals per treatment
P < 0.05 = significant
P < 0.10 = tendency
Corrected relative fluorescence of tight junction proteins CLDN3 and CLDN7 were significantly increased in ELA-treated pigs where CLDN3 was 3-fold higher and CLDN7 was 2-fold higher (P < 0.05; FIG. 10).

### CONCLUSIONS

Enhanced liquid aspirin (WO2016102959A1) contains a salicylate compound, glycerin, triacetate, and saccharin which is more stable and negates or reduces the negative effects on the GI tract than plain aspirin.

Aspirin is mainly thought to impact inflammatory signaling, exerting therapeutic effects though inhibition of cyclooxygenase isoform 2 (COX2) which produces prostaglandins and thromboxane precursors (Brzozowska & Calka, 2023). There were no differences in tissue cytokine concentrations between treatments which was not surprising in healthy non-disease, non-challenged pigs. This indicated that the ELA on its own did not increase these inflammatory markers.

Traditional forms of aspirin have been shown to induce intestinal injury and inflammation (Lin et al., 2024). Intriguingly, there were some differences in gene abundance of inflammatory-related genes including an increase in IL1 RN in ELA-treated pigs as well as decreases in IFNG and IL1B gene abundance. IL1 RN functions to inhibit activities of interleukin 1 alpha and interleukin 1 beta (Lennard, 2017) and is involved in the regenerative response in the intestinal epithelium and plays a role in maintenance of the intestinal stem cell niche (Cox et al., 2021).

In the current study, animals who received ELA gained over 1.7 times more weight than their control counterparts which was nearly 0.25 kg more in such a short period of time, 5-d treatment period. This demonstrates a significant impact on weight gain of ELA in treated animals.

This result is further supported by prior research which has shown that villus height is a good indicator of growth performance in piglets based on mucosal enzyme activities and nutrient transporters (Wang et al., 2020). Villus height shows a strong tendency towards increasing in ELA-gavaged pigs (P=0.06), indicating enhanced intestinal maturation and increased surface area for absorption (Kwon et al., 2020). This result supports the finding that the ELA composition demonstrates a growth-promoting impact on treated animals.

CLDN2 and CLDN4 gene expression were significantly higher in ELA-gavaged pigs while CLDN3 and CLDN7 protein expression was higher. Tight junction permeability can be divided into multiple pathways, specifically a pore and a leak pathway. The pore pathway allows small charge-selective molecules to enter while larger molecules utilize the leak pathway (Oami et al., 2024). Claudin-2 is a pore-forming tight junction protein highly expressed in intestinal crypts which allows for the passage of water and sodium (Monaco et al., 2021). Claudin-2 is highly expressed in intestinal crypts and stem-cell rich organoids (Pearce et al., 2018). Claudins-3 and -4 are part of the epithelial leak-pathway protein which has a function of maintaining the intestinal barrier as well as reconstitution (Gunzel & Yu, 2013; Lu et al., 2013). Claudin-7 is a TJ protein that can act as an anion pore or cation barrier in kidney but also contributes to the intestinal epithelial barrier (Garcia-Hernandez et al., 2017). More recent research shows that loss of claudin-7 leads to susceptibility of inflammation (Wang et al., 2024) and is also involved in regulating intestinal stem cell functions through Wnt and Notch pathway signaling (Xing et al., 2020)

LGR5 and SOX9 which are well known intestinal stem cell markers were also trending higher in ELA-gavaged pigs. LGR5 is a specific stem cell marker for LGR5+stem cells which are the precursors for all known intestinal epithelial cell types. SOX9 on the other hand is a general crypt cell marker which shares some overlap with Paneth cells and transit-amplifying cells along with stem cells. (Schaaf et al., 2023).

Overall, this would indicate increased cell proliferation in the ileum which would help with growth and maturation of the intestine. Again, this result supports the finding that administering the ELA composition promotes growth in animals.

Interestingly, the pathways which help determine cell fate in the intestine, namely WNT and NOTCH, were also impacted by ELA-administration. Pigs gavaged with ELA had decreased gene expression of NOTCH1 and WNT3A (Fre et al., 2009). As mentioned above, there is an important interaction of these pathways with TJ protein CLDN7 and downstream targets such as LGR5. Simultaneous downregulation of both WNT and NOTCH may lead to increased secretory lineages (Wang et al., 2024), however there was no indication of that in the present study.

In the current study, ELA was well tolerated and was able to promote weight gain and improve intestinal function over a 5-day period. This shows that ELA presents a promising option for use in animals to promote growth and productivity.

Furthermore, the study shows that ELA had positive impacts on gene abundance of inflammatory markers and cellular proliferation markers in a 5-day period, indicating that ELA presents a promising replacement for standard aspirin.

### REFERENCES

A number of publications are cited above in order to more fully describe and disclose the invention and the state of the art to which the invention pertains. Full citations for these references are provided below. The entirety of each of these references is incorporated herein.
1. Lan et al. “Antitumor effect of aspirin in glioblastoma cells by modulation of β-catenin/T-cell factor-mediated transcriptional activity”, J Neurosurg, 2011, Vol. 115, pp. 780-788;
2. M. W. Brown, ‘Characterisation Of The Effects Of Chronic Aspirin Treatment On The Viability And Proliferation Of Stage 4 Glioblastoma Cells’, Diffusion: the UCLan Journal of Undergraduate Research, Vol. 6, Issue 2, December 2013;
3. Aas et al., ‘Growth inhibition of rat glioma cells in vitro and in vivo by aspirin’, Journal of Neuro-Oncology, 1995, Vol. 24, Issue 2, pp. 171-180;
4. Ning et al., ‘Overexpression of S100A9 in human glioma and in-vitro inhibition by aspirin’, European Journal of Cancer Prevention, 2013, Vol. 22, Issue 6, pp. 585-595;
5. Hwang et al., ‘Effect of aspirin and indomethacin on prostaglandin E2 synthesis in C6 glioma cells’, Kaohsiung J Med Sci, 2004, Vol. 20, pp. 1-5;
6. Okada et al., ‘Integration of epidemiology, immunobiology, and translational research for brain tumors’, Ann N Y Acad Sci, 2013, Vol. 1284, pp. 17-23;
7. Rothwell et al., ‘Effect of daily aspirin on long-term risk of death due to cancer: analysis of individual patient data from randomised trials’, Lancet, 2011, Vol. 377, pp. 31-41;
8. K. D. Rainsford, ‘Aspirin and Related Drugs’, 2004 (book);
9. Douthwaite and Lintott, ‘Gastroscopic observations of the gastric mucosa after use of aspirin’, Lancet, 1938, Vol. 232, pp. 1222-1225;
10. Hurst and Lintott, ‘Aspirin as a cause of hematemesis’, Guy's Hosp Rep, 1939, p. 173;
11. D. J. Levy ‘An aspirin tablet and gastric ulcer’, N Engl J Med, 2000, Vol. 343, No. 12, p. 863 (photograph);
12. Rainsford et al. ‘Electronmicroscopic observations on the effects of orally administered aspirin and aspirin-bicarbonate mixtures on the development of gastric mucosal damage in the rat’, Gut, 1975, Vol. 16(7), pp. 514-527;
13. Jaiswal et al. IUPHAR 9th Int. Conference of Pharmacology, 1984;
14. Liversage et al. ‘Drug particle size reduction for decreasing gastric irritation and enhanced absorption of Naproxen in rats’, Int J Pharm, 1995, Vol. 125(2), pp. 309-313;
15. Gyory et al. ‘Effect of particle size on aspirin-induced gastrointestinal bleeding’, Lancet, Vol. 292, No. 7563, pp. 300-302;
16. M. I. Grossman et al. ‘Fecal Blood Loss Produced By Oral And Intravenous Administration Of Various Salicylates’, Gastroenterology, 1961, Vol. 40, pp. 383-388;
17. Kevin et al., ‘Acute effect of systemic aspirin on gastric mucosa in man’, Digestive Diseases and Sciences, 1980, Vol. 25, Issue 2, pp 97-99;
18. Cooke et al., ‘Failure of intravenous aspirin to increase gastrointestinal blood loss’, British medical journal, 1969; Vol. 3(5666), pp. 330-332;
19. Wallace et al., ‘Adaptation of rat gastric mucosa to aspirin requires mucosal contact’ Am J Physiol, 1995, Vol. 268, G134-8;
20. Cryer et al. ‘Effects of low dose daily aspirin therapy on gastric, duodenal and rectal prostaglandin levels and on mucosal injury in healthy humans’, Gastroenterology, 1999, Vol. 117, pp. 17-25;
21. Lichtenberger et al., ‘Where is the evidence that cyclooxygenase inhibition is the primary cause of nonsteroidal anti-inflammatory drug (NSAID)-induced gastrointestinal injury?Topical injury revisited’, Biochemical Pharmacology, 2001, Vol. 61, pp. 631-637;
22. Ligumsky M et al., ‘Aspirin can inhibit gastric mucosal cyclo-oxygenase without causing lesions in the rat’, Gastroenterology, 1983, Vol. 84, pp 756-61;
23. Zhao et al., ‘Clinical Research Feasibility of intravenous administration of aspirin in acute coronary syndrome’, Journal of Geriatric Cardiology, 2008, Vol. 5 No. 4, pp. 212-216;
24. Mashita et al., ‘Oral but not parenteral aspirin upregulates COX-2 expression in rat stomachs: a relationship between COX-2 expression and PG deficiency’, Digestion, 2006, Vol. 73(2-3), pp. 124-32;
25. Hall, SL; Lorenc, T (1 Feb. 2010). “Secondary prevention of coronary artery disease”. American family physician 81 (3): 289-96;
26. Fengming Lan et al., Antitumor effect of aspirin in glioblastoma cells by modulation of β-catenin/T-cell factor-mediated transcriptional activity. J Neurosurg 115:780-788, 2011;
27. Morris et al., Effects of Low-Dose Aspirin on Acute Inflammatory Responses in Humans. The Journal of Immunology. 2009; 183:2089-2096;
28. Gasic G I, et al. Lancet. 1972; 2:932-937;
29. Tsen et al., Triacetin-based acetate supplementation as a chemotherapeutic adjuvant therapy in glioma. Int J Cancer. 2014 March 15; 134(6):1300-1310;
30. Long et al., ‘Acetate Supplementation Induces Growth Arrest of NG2/PDGFRa-Positive Oligodendroglioma-Derived Tumor-Initiating Cells’, PLoS One, 2013, e80714;
31. Mahon et al., Saccharin: a lead compound for structure-based drug design of carbonic anhydrase IX inhibitors. Bioorg Med Chem 2015 Feb. 15; 23(4):849-54;
32. Proescholdt et al. (‘Function of carbonic anhydrase IX in glioblastoma multiforme’, Neuro Oncol, 2012, Vol. 14, pp. 1357-1366;
33. Buck M L et al., ‘Use of Aspirin in children with cardiac disease’, Pediatr Pharm, 2007, Vol. 13(1);
34. Garcia Albeniz. X et al., ‘Aspirin for the prevention of colorectal cancer’, Best Pract Res Clin Gastroenterol, 2011 August, Vol. 25(0), pp. 461-472.
35. Briggs, N. G., Silva, B. C., Godoi, L. A., & Schoonmaker, J. P. (2021). Effect of aspirin to intentionally induce leaky gut on performance, inflammation, and carcass characteristics of feedlot cattle. J Anim Sci, 99(11). https://doi.org/10.1093/jas/skab328
36. Brzozowska, M., & Calka, J. (2023). Acetylsalicylic Acid Supplementation Affects the Neurochemical Phenotyping of Porcine Duodenal Neurons. Int J Mol Sci, 24(12). 15 https://doi.org/10.3390/ijms24129871
37. Cox, C. B., Storm, E. E., Kapoor, V. N., Chavarria-Smith, J., Lin, D. L., Wang, L., Li, Y., Kljavin, N., Ota, N., Bainbridge, T. W., Anderson, K., Roose-Girma, M., Warming, S., Arron, J. R., Turley, S. J., de Sauvage, F. J., & van Lookeren Campagne, M. (2021). IL-1 R1-dependent signaling coordinates epithelial regeneration in response to intestinal damage. Sci Immunol, 6(59). https://doi.org/10.1126/sciimmunol.abe8856
38. Fre, S., Pallavi, S. K., Huyghe, M., Lae, M., Janssen, K. P., Robine, S., Artavanis-Tsakonas, S., & Louvard, D. (2009). Notch and Wnt signals cooperatively control cell proliferation and tumorigenesis in the intestine. Proc Natl Acad Sci USA, 106(15), 6309-6314. https://doi.org/10.1073/pnas.0900427106
39. Gabler, N. K., Spencer, J. D., Webel, D. M., & Spurlock, M. E. (2007). In utero and postnatal exposure to long chain (n-3) PUFA enhances intestinal glucose absorption and energy stores in weanling pigs. J Nutr, 137(11), 2351-2358. https://doi.org/10.1093/jn/137.11.2351
40. Garcia-Hernandez, V., Quiros, M., & Nusrat, A. (2017). Intestinal epithelial claudins: expression and regulation in homeostasis and inflammation. Ann N Y Acad Sci, 1397(1), 66-79. https://doi.org/10.1111/nyas.13360
41. Gunzel, D., & Yu, A. S. (2013). Claudins and the modulation of tight junction permeability. Physiol Rev, 93(2), 525-569. https://doi.org/10.1152/physrev.00019.2012
42. Helm, E. T., Curry, S. M., De Mille, C. M., Schweer, W. P., Burrough, E. R., & Gabler, N. K. (2020). Impact of viral disease hypophagia on pig jejunal function and integrity. PLoS One, 15(1), e0227265. https://doi.org/10.1371/journal.pone.0227265
43. Hofling, U., Tacconelli, S., Contursi, A., Bruno, A., Mucci, M., Ballerini, P., Cohen, S., & Patrignani, P. (2022). Characterization of the acetylation of cyclooxygenase-isozymes and targeted lipidomics of eicosanoids in serum and colon cancer cells by the new aspirin formulation IP1867B versus aspirin in vitro. Front Pharmacol, 13, 1070277. https:://doi.org/10.3389/fphar.2022.1070277
44. Kedir, H. M., Sisay, E. A., & Abiye, A. A. (2021). Enteric-Coated Aspirin and the Risk of Gastrointestinal Side Effects: A Systematic Review. Int J Gen Med, 14, 4757-4763. https://doi.org/10.2147/ijgm.S326929
45. Kwon, O., Han, T. S., & Son, M. Y. (2020). Intestinal Morphogenesis in Development, Regeneration, and Disease: The Potential Utility of Intestinal Organoids for Studying Compartmentalization of the Crypt-Villus Structure. Front Cell Dev Biol, 8, 593969. https://doi.org/i0.3389/fcell.2020.593969
46. Lambert, G. P., Schmidt, A., Schwarzkopf, K., & Lanspa, S. (2012). Effect of aspirin dose on gastrointestinal permeability. Int J Sports Med, 33(6), 421-425. https://doi.org/10.1055/s-0032-1301892
47. Lennard, A. C. (2017). Interleukin-1 Receptor Antagonist. Crit Rev Immunol, 37(2-6), 531-559. https://doi.org/10.1615/CritRevlmmunol.v37.i2-6.160
48. Li, Z., Wang, Z., Shen, B., Chen, C., Ding, X., & Song, H. (2020). Effects of aspirin on the gastrointestinal tract: Pros vs. cons. Oncol Lett, 20(3), 2567-2578. https://doi.org/10.3892/ol.2020.11817
49. Lin, Q., Zhang, B., Dai, M., Cheng, Y., & Li, F. (2024). Aspirin Caused Intestinal Damage through FXR and ET-1 Signaling Pathways. Int J Mol Sci, 25(6). https://doi.org/10.3390/ijms25063424
50. Lu, Z., Ding, L., Lu, Q., & Chen, Y. H. (2013). Claudins in intestines: Distribution and functional significance in health and diseases. Tissue Barriers, 1(3), e24978. https://doi.org/10.4161/tisb.24978
51. Monaco, A., Ovryn, B., Axis, J., & Amsler, K. (2021). The Epithelial Cell Leak Pathway. Int J Mol Sci, 22(14). https://doi.org/10.3390/ijms22147677
52. Oami, T., Abtahi, S., Shimazui, T., Chen, C. W., Sweat, Y. Y., Liang, Z., Burd, E. M., Farris, A. B., Roland, J. T., Tsukita, S., Ford, M. L., Turner, J. R., & Coopersmith, C. M. (2024). Claudin-2 upregulation enhances intestinal permeability, immune activation, dysbiosis, and mortality in sepsis. Proc Natl Acad Sci USA, 121(10), e2217877121. https://doi.org/10.1073/pnas.2217877121
53. Pavlidis, P., & Bjarnason, I. (2015). Aspirin Induced Adverse Effects on the Small and Large Intestine. Curr Pharm Des, 21(35), 5089-5093. https://doi.org/10.2174/1381612821666150915110058
54. Pearce, S. C., AI-Jawadi, A., Kishida, K., Yu, S., Hu, M., Fritzky, L. F., Edelblum, K. L., Gao, N., & Ferraris, R. P. (2018). Marked differences in tight junction composition and macromolecular permeability among different intestinal cell types. BMC Biol, 16(1), 19. https://doi.org/10.1186/s12915-018-0481-z
55. Pearce, S. C., Weber, G. J., Doherty, L. A., & Soares, J. W. (2022). Human iPSC Colon Organoid Function is Improved By Exposure to Fecal Fermentates [https://doi.org/10.1096/fba.2021-00166]. FASEB BioAdvances, n/a(n/a). https://doi.org/https://doi.org/10.1096/fba.2021-00166
56. Salichs, M., Sabate, D., Ciervo, O., & Homedes, J. (2012). Comparison of the antipyretic efficacy of ketoprofen, acetylsalicylic acid, and paracetamol, orally administered to swine. J Vet Pharmacol Ther, 35(2), 198-201. https://doi.org/10.1111/j.1365-2885.2011.01314.x
57. Schaaf, C. R., Polkoff, K. M., Carter, A., Stewart, A. S., Sheahan, B., Freund, J., Ginzel, J., Snyder, J. C., Roper, J., Piedrahita, J. A., & Gonzalez, L. M. (2023). A LGR5 reporter pig model closely resembles human intestine for improved study of stem cells in disease. Faseb j, 37(6), e22975. https://doi.org/10.1096/fj.202300223R
58. Wang, K., Liu, Y., Li, H., Liang, X., Hao, M., Yuan, D., & Ding, L. (2024). Claudin-7 is essential for the maintenance of colonic stem cell homoeostasis via the modulation of Wnt/Notch signalling. Cell Death & Disease, 15(4), 284. https://doi.org/10.1038/s41419-024-06658-x
59. Wang, M., Yang, C., Wang, Q., Li, J., Huang, P., Li, Y., Ding, X., Yang, H., & Yin, Y. (2020). The relationship between villous height and growth performance, small intestinal mucosal enzymes activities and nutrient transporters expression in weaned piglets. J Anim Physiol Anim Nutr (Berl), 104(2), 606-615. https://doi.org/10.1111/jpn.13299
60. Xing, T., Benderman, L. J., Sabu, S., Parker, J., Yang, J., Lu, Q., Ding, L., & Chen, Y. H. (2020). Tight Junction Protein Claudin-7 Is Essential for Intestinal Epithelial Stem Cell Self-Renewal and Differentiation. Cellular and molecular gastroenterology and hepatology, 9(4), 641-659. https://doi.org/10.1016/j.jcmgh.2019.12.005
61. Zaqout, S., Becker, L. L., & Kaindl, A. M. (2020). Immunofluorescence Staining of Paraffin Sections Step by Step. Front Neuroanat, 14, 582218. https://doi.org/10.3389/fnana.2020.582218

1. A method of enhancing a production trait of an animal, the method comprising administering to the animal an effective amount of a liquid composition comprising a salicylate compound, glycerin triacetate, and saccharin, wherein the salicylate compound is selected from the group consisting of aspirin, triflusal, and diflunisal, salsalate and salicylic acid, wherein the composition comprises 0.1 to 7 wt % salicylate compound and 90 to 99.9 wt % glycerin triacetate.

2. The method of claim 1, wherein the production trait is selected from growth rate, weight gain, milk production, and/or egg production.

3. The method of claim 1 or 2, wherein the animal is a non-human animal.

4. The method of claim 1, wherein the animal is a livestock animal.

5. The method of claim 1, wherein the method is a non-therapeutic method.

6. The method of claim 1, wherein the salicylate compound is aspirin.

7. The method of claim 1, wherein the composition comprises 0.1 to 5 wt % salicylate compound, optionally wherein the composition comprises 0.125 to 3 wt % salicylate compound.

8. The method of claim 1, wherein the composition comprises 94 to 99.9 wt % glycerin triacetate, optionally wherein the composition comprises 95 to 99.875 wt % glycerin triacetate.

9. The method of claim 1, wherein the composition comprises 0.125 to 3 wt % salicylate compound and 94 to 99.875 wt % glycerin triacetate.

10. The method of claim 1, wherein the composition comprises 0.1 to 3 wt % saccharin.

11. The method of claim 1, wherein the composition comprises 0 to 0.5 wt % water.

12. The method of claim 1, wherein the liquid composition further comprises a flavouring agent, optionally wherein the flavouring agent is mint oil.

13. The method of claim 1, wherein the liquid composition is free of particulates and/or the salicylate compound is completely soluble.

14. The method of claim 1, wherein the liquid composition is suitable for oral use, formulated for intravenous or intra-arterial administration, or formulated for inhalation or insufflation administration.
